(19) United States
(12) Patent Application Publication
(10) Pub. No.: US 2026/0355386 A1
(43) Pub. Date: Nov. 12, 2026

(54) BIOMATERIAL COMPOSITIONS

(71) Applicant: THE REGENTS OF THE UNIVERSITY OF MICHIGAN, Ann Arbor, MI (US)

(72) Inventors: Peter X. Ma, Ann Arbor, MI (US); Renny T. Franceschi, Ann Arbor, MI (US); Tongqing Zhou, Ann Arbor, MI (US); Chunxi Ge, Ann Arbor, MI (US)

(21) Appl. No.: 19/486,949

(22) PCT Filed: May 23, 2024
(86) PCT No.: PCT/US2024/030740

§ 371 Date: Nov. 23, 2025

Related U.S. Application Data
(60) Provisional application No. 63/504,086, filed on May 24, 2023.

(51) Int. Cl. A61L 27/22; A61L 27/26

(52) U.S. Cl. CPC A61L 27/227; A61L 27/26; A61L 2300/25; A61L 2300/252; A61L 2300/438; A61L 2400/12; A61L 2430/02

(57) ABSTRACT

An example of a biomaterial composition includes abase material and a peptide attached to a surface of the base material, where the peptide is to bind to a discoidin domain receptor of a cell. The composition may be a material, or may be used to generate an implant or medical device, or may be used as a coating on a medical device.

### CROSS-REFERENCE TO RELATED APPLICATIONS

This application claims the benefit of U.S. Provisional Application Ser. No. 63/504,086, filed May 24, 2023, the content of which is incorporated by reference herein in its entirety.

### STATEMENT REGARDING FEDERALLY SPONSORED RESEARCH OR DEVELOPMENT

This invention was made with government support under DE029012, DE029465, and AR075770 awarded by the National Institutes of Health and under W81XWH-20-1-0571 and W81XWH-20-1-0572 awarded by the U.S. Department of Defense.

### CROSS-REFERENCE TO SEQUENCE LISTING

The Sequence Listing submitted herewith is hereby incorporated by reference in its entirety. The name of the file is UMJ240BPCT_2023-539-02_Sequence_Listing.xml, the size of the file is 9,564 bytes, and the date of creation of the file is May 23, 2024.

### BACKGROUND

Tissue engineering and regenerative medicine are important research areas that aim to achieve regenerative alternatives to harvested tissues for transplantation. Synthesized biomaterials have been shown to be useful for engineering tissue regeneration and repair, at least in part because they recapitulate the physical characteristics of the biological tissue environment. For example, synthetic biomaterials have been generated with physical architecture that mimics the extracellular matrix (ECM). These biomaterials have been used as drug carriers and/or tissue engineering scaffolds. However, many of the available synthetic biomaterials are not able to deliver the biochemical cues of the natural ECM. For example, many synthetic biomaterials lack reactive sites for the presentation of a desirable biochemical stimuli.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a schematic illustration of one example of a polymeric structure, in the form of a microsphere, for use in the biological composition disclosed herein;

FIG. 1B is a schematic illustration of another example of a polymeric structure, in the form of a nanofibrous film, for use in the biological composition disclosed herein;

FIG. 1C is a schematic illustration of yet another example of a polymeric structure, in the form of a scaffold, for use in the biological composition disclosed herein;

FIG. 2A is a graph depicting the mass spectrometry of the discoidin domain receptor 2 (also referred to herein as DDR2) binding peptide GVM (number average molecular weight 4,240);

FIG. 2B is a graph depicting the mass spectrometry of the integrin binding peptide GER (number average molecular weight 3,657);

FIG. 3 is a graph depicting the circular dichroism (CD) spectroscopy results for type I collagen and the GVM1 and GER peptides (showing mdeg, Y axis, versus the wavelength in nm, X axis), where the small positive peak near 220-230 nm and the large negative trough near 200 nm indicate the triple helix structure;

FIG. 4A is a graph depicting the amount of bound peptide (μg/cm², Y axis) versus the coating concentration (μg/mL, X axis) for tissue culture plates coated with type I collagen or modified GVM (referred to herein as GVM1, see SEQ. ID. NO. 4);

FIG. 4B is a graph depicting the amount of bound peptide (μg/cm², Y axis) versus the coating concentration (μg/mL, X axis) for tissue culture plates coated with GVM1 or GER (referred to herein as GER, see SEQ. ID. NO. 5);

FIG. 5 is a graph depicting the percentage of bound peptide (%, Y axis) versus the number of days (X axis) that had passed since the tissue culture plates were coated with GVM1;

FIG. 6A is a graph depicting the ratio of pDDR2/tDDR2 (Y axis) versus the coating concentration (μg/mL, X axis) of the GVM1 peptide or type I collagen;

FIG. 6B is a graph depicting the ratio of pFAK/tFAK (Y axis) versus the coating concentration (μg/mL, X axis) of the GER peptide;

FIG. 6C is a graph depicting the ratio of pDDR2/tDDR2 (Y axis) versus the coating concentration (μg/mL, X axis) of the GER peptide;

FIG. 7A and FIG. 7B are bar graphs depicting the selective activities on pFAK or pDDR2, respectively, where the Y axis depicts the average corrected total cell fluorescence (CTCF) obtained from immunofluorescence microscopy images of MC3T3E1 cells plated on the control and the GER, GVM1, and combined GER-GVM1 peptide-coated tissue culture plates (each of which is identified on the X axis) (data are presented as mean+SD for each group, and a 4-way ANOVA was used to assess significance between groups, *p<0.01, **p<0.001);

FIG. 8 is a bar graph comparing cell attachment in the presence or absence of peptides (means and SD (error bars) are shown for triplicate samples (p<0.05));

FIG. 9A through FIG. 9C are bar graphs respectively depicting the ratio of Ddr2/Gapdh (9A), Alpl/Gapdh (9B), and Bglap/Gapdh (9C) for tissue culture plates coated with type I collagen or GVM1 at different times during incubation (data are presented as mean+SD for each group, n=3, and a two-tailed unpaired t-test was used to analyze the difference between the controls and GVM1 peptide-treated groups, *p<0.01, **p<0.001);

FIG. 10 is a bar graph comparing the DDR1, DDR2, Vwf and SPARC expression in bone cells (MC3T3-E1cl14, BMSCs and primary calvarial osteoblasts plated on tissue culture plates coated with GVM1) after a 24-hour attachment period (means and SD (error bars) are shown for triplicate samples);

FIG. 11 a bar graph depicting the concentration of Alizarin Red (μg/dish, Y axis) for each of: an Alizarin Red stained control sample (MC3T3E1 cells seeded on an uncoated tissue culture), an Alizarin Red stained GVM1 sample (MC3T3E1 cells seeded on a GVM1 peptide coated tissue culture), an Alizarin Red stained GER sample (MC3T3E1 cells seeded on a GER peptide coated tissue culture), and an Alizarin Red stained GVM1+GER sample (MC3T3E1 cells seeded on a combined GVM1-GER peptide coated tissue culture) after 2 weeks of cell incubation;

FIG. 12A through FIG. 12D are graphs respectively depicting the ratio of AlpI/Gapdh (12A), Ibsp/Gapdh (12B), Bglap/Gapdh (12C), and Ddr2/Gapdg (12D) for tissue culture plates without coating (CTR) or coated with GVM1, GER, or GVM1+GER and seeded with MC3T3E1 cells at different times during incubation (data are presented as mean+SD for each group, n=3; a 4-way ANOVA was used to separately assess differences between groups at day 10 or day 14; “a” identifies where the GVM1 peptide significantly differed from the control, p<0.01; and “b” identifies where the GVM1+GER peptide significantly differed from the GVM1 peptide, p<0.01;

FIG. 13 is a bar graph depicting the concentration of Alizarin Red (μg/dish, Y axis) for each of: an Alizarin Red stained control sample (murine bone marrow stromal cells (BMSCs) seeded on an uncoated tissue culture), an Alizarin Red stained GVM1 sample (BMSCs seeded on a GVM1 peptide coated tissue culture), an Alizarin Red stained GER sample (BMSCs seeded on a GER peptide coated tissue culture) and an Alizarin Red stained GVM1+GER sample (BMSCs seeded on a combined GVM1+GER peptide coated tissue culture) after 3 weeks of cell incubation;

FIG. 14A through FIG. 14C are bar graphs respectively depicting the ratio of Ddr2/Gapdg (14A), Bglap/Gapdh (14B), and Ibsp/Gapdh (14C), for tissue culture plates without coating (CTR) or coated with GVM1, GER, or GVM1+GER and seeded with BMSCs (data are presented as mean+SD for each group, n=3, and a 4-way ANOVA was used to separately assess differences between groups, *p<0.01, **p<0.001);

FIG. 15A is a bar graph depicting the ratio of pDDR2/tDDR2 (Y axis) for control samples (adLacZ-treated) and adCre-treated primary calvaria osteoblasts cells seeded on tissue culture plates without coating (CTR) or coated with GVM1, GER, or combined GVM1 and GER peptides, where DDR2 phosphorylation was measured after 8 hours;

FIG. 15B is a bar graph depicting the ratio of pFAK/tFAK (Y axis) for control samples (adLacZ-treated) and adCre-treated primary calvaria osteoblast cells seeded on tissue culture plates without coating (CTR) or coated with GVM1, GER, or combined GVM1 and GER peptides, where FAK phosphorylation was measured after 8 hours;

FIG. 16 is a bar graph depicting the concentration of Alizarin Red (μg/dish, Y axis) for each of control samples (adLacZ-treated) and adCre-treated primary calvaria osteoblast cell samples;

FIG. 17A through FIG. 17C are bar graphs respectively depicting the ratio of Bglap/Gapdh (17A), Ibsp/Gapdh (17B), and Alpl/Gapdh (17C) for each of control samples (adLacZ-treated) and adCre-treated primary calvaria osteoblast cell samples (data are presented as mean+SD for each group, and a 4-way ANOVA was used to separately assess differences between groups, *p<0.01, **p<0.001);

FIG. 18 schematically depicts the chemical reaction scheme for poly(HEMA-graft-PLLA)-methacrylate (i.e., PHEMA-g-PLLA-ma);

FIG. 19A is the H NMR spectrum of the HEMA-PLLA macromonomer;

FIG. 19B is the H NMR spectrum of PHEMA-g-PLLA-ma;

FIG. 20 is a graph depiction of the Fourier Transform Infrared (FTIR) Spectroscopy results for (a) HEMA-PLLA, (b) PHEMA-g-PLLA, (c) PHEMA-g-PLLA-ma, and (d) a nanofibrous PHEMA-g-PLLA-ma film after peptide conjugation;

FIG. 21 depicts several Scanning Electron Microscopy (SEM) images of films made with PLLA (0/100) or blends of PHEMA-g-PLLA-ma/PLLA at various weight ratios (20/80, 40/60, 50/50, 60/40, and 80/20);

FIG. 22A is a bar graph of average fiber diameter (nm, Y axis) for films made with PLLA or blends of PHEMA-g-PLLA-ma:PLLA at various weight ratios (0/100, 20/80, 40/60, 50/50) (n=100);

FIG. 22B is a bar graph of strain at break (%, Y axis) for the films made with PLLA or blends of PHEMA-g-PLLA-ma:PLLA at various weight ratios (0/100, 20/80, 40/60, 50/50) (n=5, *P<0.05);

FIG. 22C is a bar graph of Young's Modulus (MPa, Y axis) for the films made with PLLA or blends of PHEMA-g-PLLA-ma:PLLA at various weight ratios (0/100, 20/80, 40/60, 50/50) (n=5);

FIG. 23 is a bar graph depicting the peptide conjugation percentage (%, Y axis) for nanofibrous films blends of PHEMA-g-PLLA-ma:PLLA at various weight ratios (10/90, 20/80, 40/60, 50/50) (n=3, **P<0.01);

FIG. 24A is a graph depicting the amount of conjugated peptide per mass of 3D scaffold (μg peptide/mg scaffold, Y axis) vs the feed peptide per mass or 3D scaffold (μg peptide/mg scaffold, X axis) (n=3);

FIG. 24B is a graph depicting the percentage of conjugated peptide (%, Y axis) with a changing peptide feed (μg peptide/mg scaffold, X axis) (n=3);

FIG. 25 depicts black and white reproductions of fluorescent microscope images of nanofibrous films with labeled peptides attached via physical absorption or covalent conjugation, where the images were taken at different time points after immersion in phosphate buffered saline (PBS) at 37 °C;

FIG. 26A depicts black and white reproductions of microscope images of H&E stained nanofibrous PLLA-g-HEMA scaffolds with or without GFOGER/GVM peptides, and at different time periods following incubation;

FIG. 26B and FIG. 26C are graphs depicting the DNA counting of multi-potent periodontal ligament (PDL) cells per scaffold (1*10⁶ cells/scaffold) after overnight and 7-day incubation periods;

FIG. 27 is a bar graph depicting the tensile modulus (MPa, Y axis) of nanofibrous films formed with a 50/50 blend of pure PLLA and graft copolymers with different PLLA chain length (identified on the X axis);

FIG. 28 is a bar graph depicting the conjugation density (nmol peptide per mg scaffold, Y axis) of nanofibrous scaffolds formed with a 50/50 blend of pure PLLA and graft copolymers prepared with different PLLA chain length (identified on the X axis) and different HEMA/L-lactide feed ratios;

FIG. 29 is a bar graph depicting the conjugation density (nmol peptide per mg scaffold, Y axis) of nanofibrous scaffolds formed with a 50/50 blend of pure PLLA and the graft copolymers with different PLLA chain length (identified on the X axis);

FIG. 30 depicts black and white reproductions of fluorescent microscope images of nanofibrous scaffolds with different ratios of two different labeled peptides (GER3-Avidin-Alex dye:GMV2-FITC dye) attached via covalent conjugation (scale bar=100 μm, 4:0 (originally red), 3:1 (originally lighter red), 2:2 (originally yellowish), 1:3 (originally green) and 0:1 (originally bright green));

FIG. 31 is a graph depicting the percentage of weight remaining (%, Y axis) versus time (weeks, X axis) for an example nanofibrous scaffold (PHEMA-g-PLLA-ma/PLLA) and a comparative scaffold (PLLA);

FIG. 32 are Micro CT images, reproduced herein in black and white, of critical-sized calvarial bone defect repair with different cell-free 3D scaffold implantations (control, GER2 peptide, GVM2 peptide, GER2+GVM2 peptides) after 4 and 8 weeks;

FIG. 33 is a graph depicting the bone volume (mm³) for the different cell-free 3D scaffold implantations (one-way ANOVA was used to access significance from multiple comparison, *p≤0.05, **p≤0.01); and

FIG. 34 is a schematic illustration of a hip joint including a coating of the biological composition described herein.

### SUMMARY

The biological compositions disclosed herein include a peptide, which is capable of binding to a discoidin domain receptor of a cell, attached to a surface of a base material. The triple helical peptide activates discoidin domain receptors (DDRs), which can stimulate DDR2 phosphorylation and activation with kinetics similar to native collagen. As such, the peptide contributes to enhanced tissue regeneration. The base material may be a variety of polymers or a hydrogel. Thus, the biological composition may be used to manufacture implants and medical devices. The biological composition may also be used as a coating on at least a portion of a medical device.

In some specific examples, the biological composition disclosed herein includes two peptides, namely i) the peptide that is capable of binding to a discoidin domain receptor of a cell and ii) a second peptide that is capable of binding to an integrin. In other words, the base material is functionalized with a combination of triple helical peptides that can respectively activate discoidin domain receptors (DDRs) and integrins. The combination of triple helical peptides activates certain cellular activities, which leads to enhanced tissue regeneration. In particular, this combination of triple helical peptides cooperatively interacts and synergistically stimulates osteoblast differentiation of skeletal progenitor cells (SPCs) and mineralization. Interactions between SPCs and their collagenous extracellular niche are important for bone, cartilage, and various joint development and regeneration. With the combination of triple helical peptides disclosed herein, the biological compositions are able to mediate cell-extracellular matrix (ECM) interactions, which in turn enhances tissue regeneration. Thus, these biological compositions are particularly suitable for use in a variety of applications in human healthcare, including as implants, as drug/biomolecule delivery systems, in medical devices, and in diagnostic equipment.

### DETAILED DESCRIPTION

Biological compositions are disclosed herein that are particularly useful for bone development and regeneration. The biological compositions include a base material and a synthetic triple-helical peptide attached thereto. The synthetic triple-helical peptide is capable of binding to a discoidin domain receptor of a cell. In some examples, this peptide is used in combination with a second peptide, which is capable of binding integrins.

In some instances, the base material of the biological composition is manufactured into a construct, such as a scaffold, film, or microsphere. These constructs may be used as implants. It may be desirable, in these instances, for the architecture of the base material to mimic the extracellular matrix in both fiber diameter and geometry, which enhances bone regeneration. In some of the biological compositions disclosed herein, two synthetic triple-helical peptides are used that mimic native collagen peptides and activate the two main collagen receptors in bone cells, namely collagen-binding integrins and discoidin domain receptors. The biological compositions provide biocompatibility, biodegradability, osteogenic activity, adequate mechanical properties to maintain the three-dimensional shape for bone regeneration, and a porous structure to enhance vascularization and tissue regeneration.

As mentioned, in some examples, the biomaterial compositions disclosed herein include the base material (e.g., a polymeric structure); and a discoidin domain receptor binding triple helical peptide attached to a surface of the base material. Other examples also include an integrin binding triple helical peptide attached to a surface of the base material.

The base material may be made from any material that includes a plurality of a functional group that is capable of attaching to the triple helical peptide(s). The attachment may be covalent bonding, ionic bonding, or hydrogen bonding, or Van der Walls interactions. The functional group is selected from the group consisting of double bonded carbons, triple bonded carbons, hydroxyl, aldehyde, carboxyl, isocyanate, acrylate, and methacrylate.

The base material is selected from the group consisting of a polymer and a hydrogel.

The polymer (i.e., polymeric material) may be selected from natural or synthetic hydrophilic polymers, natural or synthetic hydrophobic polymers, natural or synthetic amphophilic polymers, degradable polymers, non-degradable polymers, partially degradable polymers, proteins, polysaccharides, hydrocarbon polymers, lipids, artificial proteins, and/or combinations thereof. The polymeric material may be a homopolymer, a copolymer, or mixtures of polymers.

In one example, the polymer base material is a non-degradable polymer selected from the group consisting of polyethylene terephthalate (PET), polystyrene, a silicone polymer, a polyurethane, polyetherether ketone (PEEK), a polyamide, polycarbonate, polytrimethylene carbonate (PTMC), and mixtures thereof. These materials may be particularly suitable for medical device applications.

In another example, the polymer base material is a biodegradable polymer selected from the group consisting of poly(L-lactic acid) (PLLA), polyglycolic acid (PGA), poly(lactide-co-glycolide) (PLGA), poly(D,L-lactic acid) (PDLLA), polyanhydrides, poly(ortho ethers), poly(ε-caprolactone) (PCL), poly(hydroxy butyrate) (PHB), poly(propylene fumarate) (PPF), polyphosphoesters (PPE), polyphosphazenes, polycarbonates, polyethylene glycol (PEG), polyvinyl alcohol (PVA), polyurethane, collagen, gelatin, elastin, alginate, chitin, chitosan, pectin, copolymers thereof, and mixtures (blends) thereof.

In some examples, the polymer (i.e., homopolymer, copolymer, or polymer blend) is selected so that the polymer structure has a nanofibrous architecture (i.e., is nanofibrous), which resembles the ECM. In this example, the walls of the polymer structure are made up of interconnected nanofibers, where each nanofiber has a diameter ranging from about 1 nm to about 1000 nm. In other examples, the walls of the polymer structure are solid with some pores formed therein. Examples of polymer that can form non-nanofibrous materials include PLGA, PDLLA, and other copolymers.

One example of a nanofibrous forming copolymer that is also capable of attaching the triple helical peptide(s) is poly(hydroxyethyl-methacrylate)-graft-poly(L-lactide)-methacrylate (i.e., PHEMA-g-PLLA-ma or poly(HEMA-graft-PLLA)-methacrylate) having a hydroxyethyl-methacrylate (HEMA) content ranging from about 1 wt % to about 15 wt %. A higher percentage of HEMA may be used, although the architecture may be less nanofibrous as the HEMA content increases, e.g., when phase separation is used to form the base material. In one example, the base material is made up of poly(hydroxyethyl-methacrylate)-graft-poly(L-lactide)-methacrylate having a hydroxyethyl-methacrylate ranging from 1 wt % to 99 wt %. Some blends of polymers and/or copolymers provide the polymer base material with the nanofibrous architecture and the ability to attach the triple helical peptides. As examples, the nanofibrous polymer base material includes a blend of i) a nanofibrous-forming polymer selected from the group consisting of poly(L-lactic acid), polycaprolactone, poly(lactide-co-glycolide) with either less than 15% lactide or less than 15% glycolide, poly(D,L-lactic acid), and combinations thereof; and ii) a peptide conjugation polymer which contains the functional group for triple helical peptide conjugation. In one specific example, the nanofibrous polymeric structure is made up of a blend of PHEMA-g-PLLA-ma and poly(L-lactide) at a weight ratio ranging from 10:90 to 50:50. If a less nanofibrous structure is desired, the blend of PHEMA-g-PLLA-ma and poly(L-lactide) may be used at a weight ratio ranging of 55:45 to 95:5.

In another example, the base material is made up of a hydrogel. As an example, the hydrogel may be formed of a water-soluble polymer, which includes a repeating unit and a pendant chain covalently attached to the repeating unit. Generally, the water-soluble polymer may be a modified dextran, a modified poly(vinyl alcohol), a modified chitosan, a modified cellulose, or some other water-soluble polymer that is modified with the pendant group disclosed herein. The water-soluble polymer is a multi-functionalized polymer because of the pendant groups attached to each repeating unit. The repeating unit has at least one functional group that is capable of reacting with an isocyanate or with another functional group that is attached to an isocyanate in order to covalently bind the pendant group (which includes the isocyanate) thereto. Examples of the repeating unit include glucose unit(s), vinyl alcohol, D-glucosamine, two β(1→4) linked D-glucose units, or another repeating unit that forms a water-soluble backbone and include a suitable functional group for covalently attaching the pendant chain. The pendant chain includes ureido-pyrimidinone.

Alternatively, it is believed that the base material can be made up of macromolecules or their segments, which are derived from natural beings, such as humans, animals, or plants.

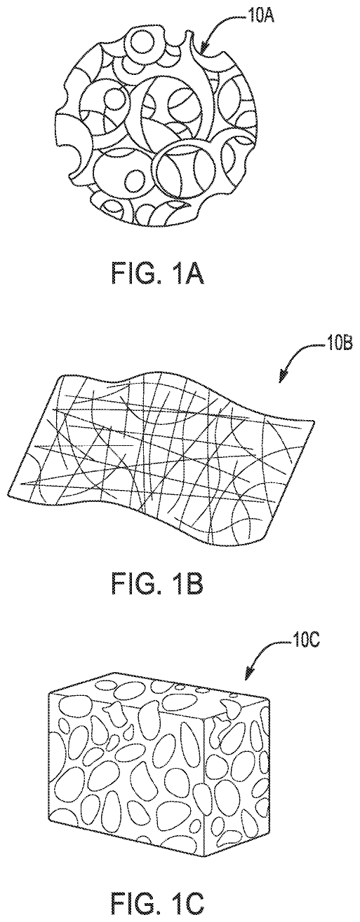

When the polymer is selected as the base material, the polymer may be in the form of a microsphere 10A (see FIG. 1A), a film 10B (see FIG. 1B), a scaffold 10C (see FIG. 1C), or another implantable medical device.

The microsphere 10A may be a nanofibrous hollow microsphere (NF-HMS), a nanofibrous microsphere (NF-MS), or a nanofibrous spongy microsphere (NF-SMS).

A nanofibrous hollow microsphere (NF-HMS) is characterized as a hollow structure having a single hollow core surrounded by a nanofibrous shell, and one or more openings formed in the nanofibrous shell. The entire hollow structure has a diameter ranging from about 5 μm to about 1000 μm. The diameter of the opening in the center of the nanofibrous shell ranges from about 5 μm to about 50 μm. The nanofibrous shell 12 include nanofibers and spaces (less than 2 μm in diameter) that are present between the nanofibers.

A nanofibrous microsphere (NF-MS) is characterized as a structure composed of nanofibers. The NF-MS includes spaces (less than 2 μm in diameter) between the nanofibers, but does not include any larger openings. The entire structure has a diameter ranging from about 5 μm to about 1000 μm.

Spongy microspheres also include a nanofibrous architecture, and have a diameter D ranging from about 5 μm to about 1000 μm. These microspheres are also spongy (i.e., nanofibrous spongy microspheres or NF-SMS). By “spongy,” it is meant that the NF-SMS have a sponge-like architecture throughout the entirety of the microsphere. The sponge-like architecture includes interconnected porous walls and micro-scale pores formed among the interconnected porous walls. The spongy microsphere is schematically depicted in FIG. 1A, although the fibrous architecture of the microsphere walls is not shown for clarity.

FIG. 1B depicts an example of the film 10B, which is made up of a plurality of nanofibers. The film 10B is a thin layer having dimensions suitable for the application in which it is to be used. The thickness of the film may range from about 1 nm to about 1 mm.

FIG. 1C depicts an example of the scaffold 10C. The scaffold 10C is formed of a plurality of nanofibers aggregated together and pores defined between at least some of the nanofibers. In FIG. 1C, the fibrous architecture of the scaffold walls is not shown for clarity. The nanofibrous scaffold is characterized as a multi-level porous structure with regular spherical macro-scale pores (ranging from about 250 μm to about 425 μm in diameter), micro-scale interpore openings (i.e., openings that connect one macro-scale pore to another macro-scale pore) of about 100 μm, and spaces (less than 2 μm across) between the nanofibers. While the pores of the scaffold are on the macro-scale or smaller, the scaffold itself has larger dimensions. For example, the thickness of the scaffold may be 0.2 mm or more, and the length and/or width of the scaffold may be 3 mm or more.

The biological composition also includes the peptide that is capable of binding to a discoidin domain receptor. This peptide may be referred to herein as a “discoidin domain receptor binding triple helical peptide.” In some examples, the discoidin domain receptor binding triple helical peptide is used alone. In other examples, the discoidin domain receptor binding triple helical peptide is used in combination with the peptide that is capable of binding to an integrin. This second peptide may be referred to herein as an “integrin binding triple helical peptide”. The peptides are able to interact with cell-surface receptors and integrins, respectively, to activate certain biological activities. More specifically, the peptides can respectively interact with discoidin domain receptors (DDRs) and with integrins.

The discoidin domain receptor binding peptide is based on GVMGFO (O=4-hydroxyproline or “4Hyp”) (SEQ. ID. NO. 1), which is present in fibrillar collagens. In the examples set forth herein, norleucine (Nle) is substituted for the methionine (M) present in the native GVMGFO sequence in order to increase stability. As such, in the examples set forth herein, the discoidin domain receptor binding peptide has the basic amino acid sequence: GPRGQOGV(Nle)GFO (SEQ. ID. NO. 2). This sequence is the core sequence, and can be flanked on either end with spacers or amino acids or linkers that serve as a reaction site functional group to attach to the base material (examples of which include the polymeric structures 10A, 10B, or 10C). In an example, the flanking amino acids include one or more GPP amino acid sequences with a GPC sequence at each end.

The discoidin domain receptors (DDR1, DDR2) are a family of collagen receptors having unique functions in bone. DDRs are distinct from integrins in that they contain intrinsic tyrosine kinase activity, but unlike classic receptor tyrosine kinases (such as growth factor receptors), DDRs are activated and phosphorylated by fibrillar collagens with an unusually slow time course. DDRs do not bind integrins or directly stimulate integrin activity, but rather increase integrin-mediated downstream signaling.

DDR1 and DDR2 both bind to the core sequence, GV(Nle)GFO, the native sequence of which is present in the COL1A1, COL2A1 and COL3A1 chains of types I, II and III collagens. This sequence is also recognized by other collagen-binding proteins such as von Willebrand Factor (vWF), a serum protein involved in blood coagulation, and Secreted Protein Acidic and Rich in Cysteine (SPARC), a secreted protein involved in collagen mineralization. In the examples disclosed herein, the GV(Nle)GFO sequence must be in a triple-helical conformation to bind DDRs, with receptor recognition requiring amino acid residues on 2 strands of the collagen helix. Triple-helical collagen-like peptides containing the GV(Nle)GFO sequence, when adsorbed to tissue culture surfaces, can stimulate DDR2 phosphorylation and activation with kinetics similar to native collagen. The DDR-activating triple-helical peptide, while not able to directly affect integrin activity, enhances integrin-dependent cell adhesion to intermediate affinity integrin-binding peptides.

As mentioned, in some instances, the discoidin domain receptor binding triple helical peptide is used in combination with the integrin binding triple helical peptide.

The integrin binding peptide has the basic amino acid sequence: GFOGER (SEQ. ID. NO. 3), which binds β1 integrins (a1β1, a2β1, a10β1, a11β1). In this sequence, O is again “4Hyp” or 4-hydroxyproline. The GFOGER sequence is the core sequence, and can be flanked on either end with spacers or amino acids or linkers that serve as a reaction site functional group to attach to the base material. In an example, the flanking amino acid is one or more GPP amino acid sequences. Synthetic triple-helical peptides containing the GFOGER sequence can stimulate osteoblast differentiation of preosteoblast cell lines and bone marrow stromal cells. Synthetic triple-helical peptides containing the GFOGER sequence can also increase cell adhesion and focal adhesion kinase (FAK) Y397 phosphorylation.

Some specific examples of the triple-helical peptides are shown herein in SEQ. ID. NOS. 4-6. The peptides may be synthesized or derived from natural materials, such as collagen or other extracellular matrix components.

The inclusion of the distinct triple helical peptide or combination of peptides allows the biological compositions to activate the discoidin domain receptors (DDR1 and DDR2), or the combination of DDRs and the collagen-binding integrins, which function as collagen receptors in bone. As described, each receptor is activated by the distinct (modified) collagen sequences: GV(Nle)GFO (SEQ. ID. NO.2) for DDRs AND GFOGER (SEQ. ID. NO. 3) for integrins.

The resulting biological composition may have desirable mechanical properties and a desired peptide conjugation. In one example, the base material has a tensile modulus of at least 10 kPa. In one example, the base material (e.g., the polymeric structures 10A, 10B, or 10C) has a tensile modulus of at least 40 kPa. In one example, the total conjugation of the discoidin domain receptor binding triple helical peptide and the integrin binding triple helical peptide is at least 4.4 nmol peptides per square meter of base material surface area.

In the examples disclosed herein, the peptides are attached to the base material. The attachment may be via bonding (e.g., covalent, ionic, or hydrogen) or via Van der Walls interactions. In one example, the discoidin domain receptor binding triple helical peptide alone or in combination with the integrin binding triple helical peptide is/are attached covalently to the base material. In another example, the discoidin domain receptor binding triple helical peptide alone or in combination with the integrin binding triple helical peptide is attached via physical absorption to the base material.

The biological composition may also be manufactured into a medical device. As examples, the biological composition may be used to form artificial hips and joints, and bone fixation devices for long bones, ribs and craniofacial bones, such as screws, bolts, plates, rots and the like.

In some examples, the biological composition is used as a coating on at least a portion of a medical device. As examples, the biological composition may form a coating on at least a portion of a titanium hip replacement, joint replacement, spine replacement or fixture, or on a dental implant. An example is shown in FIG. 34, where the medical device 12 includes a metal hip replacement 14 with a coating 16 of the biological composition.

One example method to form the biological composition includes attaching a peptide to a surface of a base material, wherein the peptide is to bind a discoidin domain receptor of a cell. In some examples, the method includes forming the base material; and attaching a discoidin domain receptor binding triple helical peptide, alone or in combination with an integrin binding triple helical peptide, to a surface of the base material. Attachment may be via covalent attachment (e.g., as described in Examples 2 and 3) or physical absorption (e.g., as described in Examples 2 and 3). Other attachment mechanisms, such as ionic bonding or hydrogen bonding may also be used.

In one example, the base material is a polymer, the polymer is in a form of the microsphere 10A; and forming the microsphere 10A involves an emulsion technique. The emulsion technique may involve forming a solution of the polymer in a non-aqueous solvent, and then emulsifying the solution in an aqueous solution to create an oil-in-water emulsion. The emulsion may be stirred for a predetermined time period in order to evaporate the non-aqueous solvent and form polymer microspheres. Nanofibrous hollow microspheres 22B and nanofibrous microspheres 22C may also be formed by phase separation and template leaching techniques or emulsification techniques in which glycerol is added to emulsify the polymer solution. Examples of methods that may be used to form the nanofibrous spongy microspheres are described in U.S. patent application Ser. No. 14/507,523, entitled “Nanofibrous Spongy Microspheres”, which is incorporated herein by reference in its entirety.

In another example, the base material is a polymer, the polymer is in the form of the film 10B, and forming the film 10B involves thermally induced phase separation or electro-spinning. With thermally induced phase separation, the polymer is dissolved in a solvent, and then poured into a mold and sealed. The sealed mold is frozen for a predetermined amount of time to induce phase separation. The mold is then submerged in an ice-water batch while returning it to room temperature. The solidified film is then dried.

In still another example, the base material is a polymer, and the polymer is in the form of the scaffold 10C. One example for forming the scaffold includes first generating a negative replica of a scaffold by: introducing sugar spheres and a non-solvent thereof into a mold; annealing the sugar spheres, thereby causing the sugar spheres to interconnect; and removing the non-solvent; and then generating the scaffold by: casting a polymer solution into the mold and onto the negative replica of the scaffold; performing temperature-induced phase separation of the polymer solution; and removing the negative replica of the scaffold.

In any example method, if it is desirable to form a nanofibrous base material (e.g., polymeric structure 10A, 10B, 10C), then the method further includes selecting the polymer, copolymer, or polymer blend to achieve the nanofibrous architecture. Any of the polymers, copolymers, or blend of (co)polymers described herein as being capable of forming nanofibers may be selected. The polymer, copolymer, or blend of (co)polymers should allow for the controllable attachment of the peptide(s), while also maintaining nanofiber forming properties.

In some of the examples set forth herein, the blend includes the nanofibrous-forming polymer, and a graft copolymer made up of both the nanofibrous-forming polymer and the peptide conjugation polymer. In these examples, the following factors may be adjusted to ensure both nanofiber formation and peptide attachment: molecular weight of the nanofibrous-forming polymer, chain length of the nanofibrous-forming polymer in the graft copolymer, the feed ratio of the nanofibrous-forming polymer and the peptide conjugation polymer during graft copolymer formation, and the blend ratio of the nanofibrous-forming polymer and the graft copolymer. Each of these factors can affect the nanofibrous morphology, the mechanical properties, and the peptide attachment. In addition, the graft copolymer composition (feed ratio) and graft chain length can affect the degradation rate and mechanical properties of the base material.

When the blend includes the nanofibrous-forming polymer and the graft copolymer, the method may further include preparing the blend. In one example, the blend is prepared with poly(hydroxyethyl-methacrylate)-graft-poly(L-lactide)-methacrylate (i.e., the peptide conjugation polymer) and poly(L-lactide) (i.e., the nanofibrous-forming polymer) at a weight ratio ranging from 10:90 up to 60:40.

The following are a few specific example blends prepared with these two polymers. In one example, the biological composition includes a polymeric nanofibrous structure made up of a blend of poly(hydroxyethyl-methacrylate)-graft-poly(L-lactide)-methacrylate and poly(L-lactide) at a weight ratio ranging from 10:90 to 60:40; the poly(hydroxyethyl-methacrylate)-graft-poly(L-lactide)-methacrylate has a hydroxyethyl-methacrylate/L-lactide weight ratio ranging from 0.1 to 0.9; and a number average molecular weight of the poly(L-lactide) is greater than or equal to 2,000 g/mol. In another example, the biological composition includes a polymeric nanofibrous structure made up of a blend of poly(hydroxyethyl-methacrylate)-graft-poly(L-lactide)-methacrylate and poly(L-lactide) at a weight ratio of 50:50; the poly(hydroxyethyl-methacrylate)-graft-poly(L-lactide)-methacrylate has a hydroxyethyl-methacrylate/L-lactide weight ratio of 0.86; and a number average molecular weight of the poly(L-lactide) is about 17,700 g/mol. In another example, the biological composition includes a polymeric nanofibrous structure made up of a blend of poly(hydroxyethyl-methacrylate)-graft-poly(L-lactide)-methacrylate and poly(L-lactide) at a weight ratio of 10:90; the poly(hydroxyethyl-methacrylate)-graft-poly(L-lactide)-methacrylate has a hydroxyethyl-methacrylate/L-lactide weight ratio of 0.86; and a number average molecular weight of the poly(L-lactide) is about 112,000 g/mol. In still another example, the biological composition includes a polymeric nanofibrous structure made up of a blend of poly(hydroxyethyl-methacrylate)-graft-poly(L-lactide)-methacrylate and poly(L-lactide) at a weight ratio of 50:50; the poly(hydroxyethyl-methacrylate)-graft-poly(L-lactide)-methacrylate has a hydroxyethyl-methacrylate/L-lactide weight ratio of 0.86; and a number average molecular weight of the poly(L-lactide) is about 132,000 g/mol.

After the base material is formed, the peptide(s) is/are attached to a surface of the base material. The process for attaching may result in covalent, ionic, or hydrogen bonding between the peptide(s) and the functional groups of the base material, or physical absorption (Van der Walls interactions) of the peptide(s) at the surface of the base material. When covalent attachment is desired, the method involves wetting the base material (e.g., polymeric structure 10A, 10B, 10C); exposing the wetted base material to the discoidin domain receptor binding triple helical peptide (alone or in combination with the integrin binding triple helical peptide), thereby forming a precursor structure; and exposing the precursor structure to ultraviolet light. When physical absorption is desired, the method involves incubating, at a predetermined temperature and for a predetermined time, the base material (e.g., polymeric structure 10A, 10B, 10C) in a solution containing the discoidin domain receptor binding triple helical peptide (alone or in combination with the integrin binding triple helical peptide).

In other example methods, the biological composition is formed as described herein (i.e., the peptide(s) is/are attached to the base material), and then is coated on at least a portion of a medical device. Any suitable deposition technique may be used, such as painting, spray coating, liquid deposition, or vapor deposition. Alternatively, the base material may be coated on at least a portion of the medical device, and then the peptide(s) may be attached (e.g., covalently, ionically, via hydrogen bonding, or via Van der Walls interactions) to the base material coating as described herein.

The biological compositions disclosed herein can be used in in vitro and in vivo regeneration of structural tissues such as bone, articular cartilage, fibrous cartilage, meniscus, bone/cartilage composite, ligament, tendon, cementum, dentin, enamel, temporomandibular joint (TMJ) tissues, intervertebral disc, and so on.

To further illustrate the present disclosure, examples are given herein. It is to be understood that these examples are provided for illustrative purposes and are not to be construed as limiting the scope of the present disclosure.

### EXAMPLES

The molecular weights (described as “MW”) given in the following examples were measured via gel permeation chromatography (GPC), and thus are closer to number average molecular weights than weight average molecular weights.

### Example 1

In this example, specific triple helical peptides respectively containing the GV(Nle)GFO and the GFOGER binding domains were evaluated for their ability to stimulate DDR2 and integrin signaling and osteoblast differentiation. The GV(Nle)GFO based peptide stimulated DDR2 Y740 phosphorylation and osteoblast differentiation as measured by induction of osteoblast marker mRNAs and mineralization, without affecting integrin activity. In contrast, the GFOGER based peptide stimulated focal adhesion kinase (FAK) Y397 phosphorylation, an early measure of integrin activation, and to a lesser extent osteoblast differentiation, without affecting DDR2-P. Significantly, the combination of both peptides cooperatively enhanced both DDR2 and FAK signaling and osteoblast differentiation, a response that was blocked in Ddr2-deficient cells. Additionally, all statistical analyses were performed using GraphPad Prism software (version 7, La Jolla, CA, USA). Values are presented as mean±SD. Triplicate independent samples were used. Two-tailed unpaired t-test was used for 2 group comparisons, while 4-way ANOVA was used for multiple comparisons as indicated.

### Peptide Synthesis

A DDR2 binding peptide (GVM1) and an integrin binding peptide (GER) were synthesized by New England Peptide. GVM1 had the following sequence:

(SEQ. ID. NO. 4)
GPC(GPP)₅GPRGQOGV(Nle)GFO(GPP)₅GPC

and GER had the following sequence:

(SEQ. ID. NO. 5)
GPC(GPP)₅GFOGER(GPP)₅GPC.

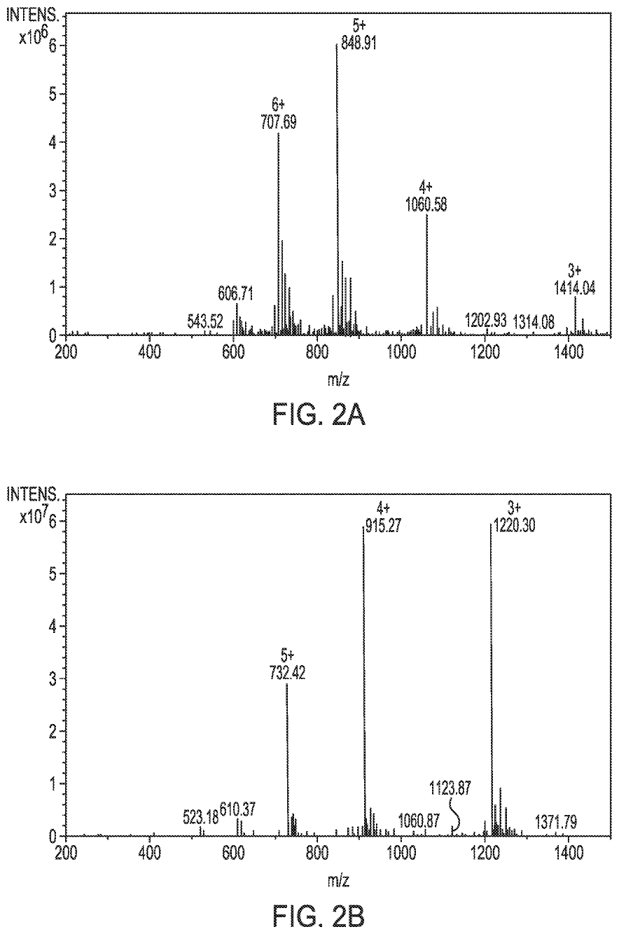

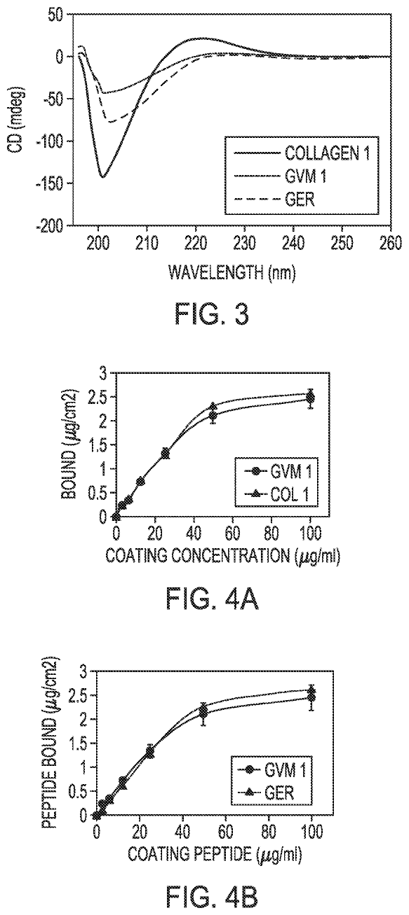

As shown in SEQ. ID. NO. 4, norleucine was substituted for the methionine present in the native GVMGFO sequence. This substitution was performed to increase stability. The mass accuracy was verified by mass spectrometry (GVM1 and GER peptide molecular weights; 4240 and 3657, respectively; see FIG. 2A and FIG. 2B). The triple-helical conformation of both peptides was confirmed using circular dichroism (FIG. 3). The peptides were dissolved in 0.1% trifluoracetic acid (TFA) at 10 mg/ml and stored at −80 °C. Coating tissue culture plates with peptides or type/collagen

The peptides or type I collagen (BD Bioscience, CB354249, as a positive control for DDR2 activation) were diluted in sterile PBS to the desired concentrations (between 1 g/mL and 100 g/mL), and 0.5 mL of the coating solutions were added into separate 12 well tissue culture plates (Corning, 07-200-81). After overnight adsorption at 4 °C, the amount of peptide or type I collagen that was bound was calculated by measuring the concentration in solution before and after coating using a Pierce Quantitative Fluorometric Peptide Assay kit (ThermoFisher, 23290).

For the GVM1 peptide and type I collagen, a coating solution concentration of 50 μg/mL saturated the plate surface to give a final coating density of 2.5 μg/cm² (FIG. 4A). The coating efficiencies of the GVM1 and GER peptides on tissue culture plates were found to be equivalent with 2.5 μg/cm², representing the maximal binding capacity for each peptide (coating solution concentration of 50 μg/ml, FIG. 4B3).

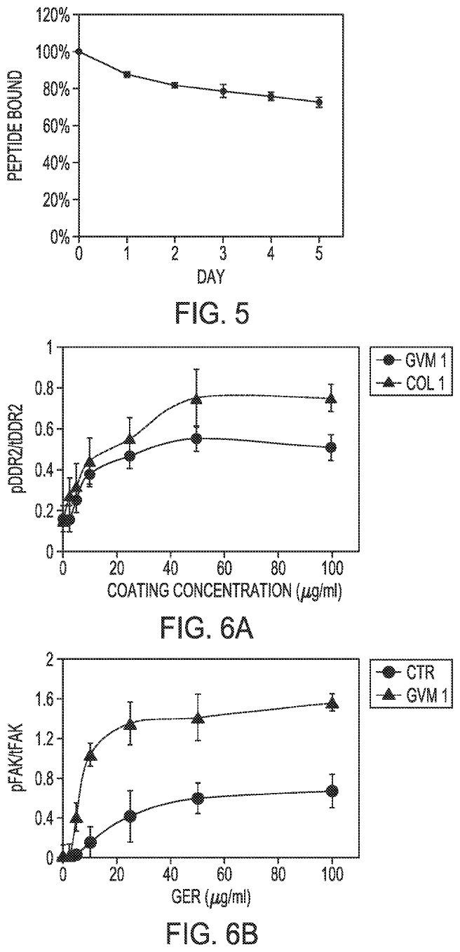

The amount of the GVM1 peptide released from the plate was measured over a 5-day period and the amount of bound peptide remaining was then calculated as a percentage of initially bound peptide. The GVM1 peptide remained bound to the plate surface for extended times as shown in FIG. 5 (data is expressed as mean+SD). After 5 days, only about 20 percent of the initially bound peptide was released.

### Cell Culture and In Vitro Differentiation

Example 1 used MC3T3E1 clone14 preosteoblast cells (referred to herein as MC3T3-E1cl14 or MC3T3E1), bone marrow stromal cells (BMSCs) isolated from C57BLJ6 mice and primary calvaria osteoblasts (COBs) isolated from Ddr2^{flox/flox} mice. For the analysis of downstream signals induced by the respective peptides and type I collagen, MC3T3-E1 cells and primary cells were seeded at a density of 50,000 cells/cm² on peptide-coated tissue culture dishes in 0.1% FBS α-MEM media and harvested at 8 hours post seeding. For in vitro differentiation, cells were grown in osteogenic medium (10% FBS α-MEM with 50 μg/mL ascorbic acid, 10 mM β-glycerophosphate) for up to 3 weeks. In some samples, Ddr2 was inactivated in COB cells by Cre mediated recombination using either lacZ (AdLacZ) or Cre-expressing adenovirus (AdCre) at an m.o.i. of 200 pfu/cell.

For Western Blot analysis, total cell lysates were collected in Tris-Glycine SDS sample buffer (Invitrogen, LC2676) and fractioned using Tris-Glycine polyacrylamide gels (Invitrogen, XP04125). The following primary antibodies were used at a 1:1000 dilution: phosphorylated DDR2 at Y740 (B&D system, MAB25382), total DDR2 (LifespanBio, LS-B15752), 21hosphor-FAK at Y397 (Cell Signaling, 3283) and total FAK (Cell Signaling 3285). Secondary antibody (horseradish peroxidase-conjugated goat anti-rabbit IgG) was used at 1:10,000 dilution, and blots were visualized using ECL substrate (Amersham). Chemiluminescence signals were detected using a ChemiDoc Imaging system (Bio-Rad, ChemiDoc MP) and analyzed using Bio-Rad software Image Lab 6.0.1.

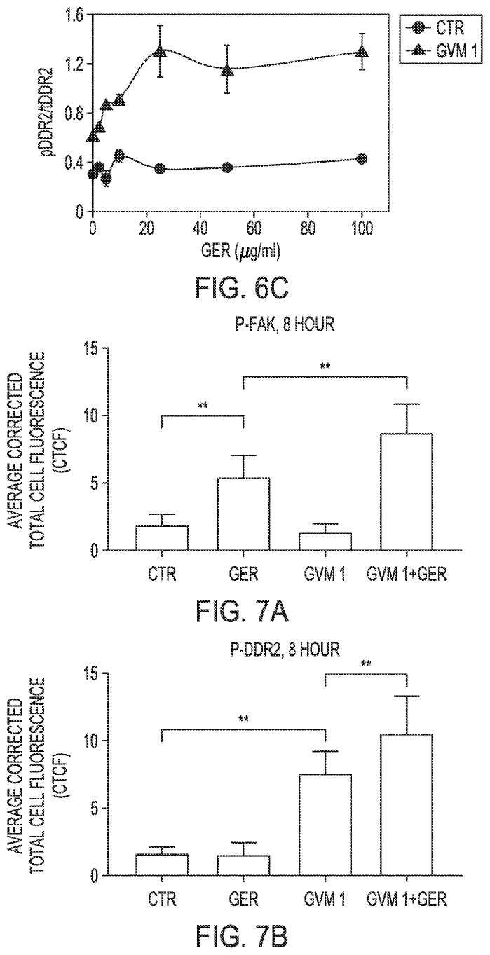

On binding collagen or synthetic peptides, DDR2 is phosphorylated on several tyrosine residues, including Y740, which is commonly used to monitor activation. To measure DDR2 activation in bone-derived cells, the MC3T3E1cl14 preosteoblast cells were seeded on the GVM1 peptide or type I collagen-coated plates, and DDR2 Y740 phosphorylation (pDDR2) was measured after 8 hours. DDR2 activation (pDDR2/totalDDR2) was measured by Western Blot (results not reproduced herein) and quantified by densitometry. The quantified results are shown in FIG. 6A. As depicted, optimum stimulation of DDR2 phosphorylation measured relative to total DDR2 (pDDR2/tDDR2) was seen at coating concentrations between 25 and 50 μg/mL for both GVM1 and type I collagen. Although total DDR2 levels remained relatively constant, the addition of GVM1 peptide or collagen increased formation of slower migrating species on SDS gels, likely as a consequence of changes in phosphorylated state.

Activation and Y397 phosphorylation of focal adhesion kinase (FAK) is an early response to integrin activation by collagen or the GER peptide. To measure interactions between the GER peptide and the DDR2-activating GVM1 peptide, MC3T3E1 cells were plated on tissue culture plates coated with concentrations of the GER peptide ranging from 5 μg/ml to 100 μg/ml, with or without 25 g/mL of the GVM1 peptide. Focal adhesion kinase (FAK) Y397 phosphorylation was measured after 8 hours. FAK Y397 activation (pFAK/tFAK) was measured by Western Blot (results not reproduced herein) and quantified by densitometry. The quantified results are shown in FIG. 6B. As shown in FIG. 6B, the GER peptide dose-dependently stimulated FAK Y397 phosphorylation relative to total FAK (pFAK/tFAK) at coating concentrations between 10 and 50 g/ml. Although the GVM1 peptide by itself did not affect pFAK, it dramatically stimulated activity of the GER peptide, particularly at lower concentrations (5-10 μg/ml GER coating concentration), where addition of the GVM1 peptide increased pFAK/tFAK up to 8-fold. For these samples, DDR2 Y740 phosphorylation (pDDR2) was also measured after 8 hours and quantified by densitometry. These results are shown in FIG. 6C. While the GER peptide did not stimulate DDR2 phosphorylation, the GER peptide dose-dependently increased pDDR2/tDDR2 in the presence of the GVM1 peptide (maximum stimulation 3-fold, FIG. 6C).

Immunofluorescence (IF) staining was used to visualize the phosphorylation of focal adhesion kinase (FAK) and DDR2 after the stimulation with collagen ligand mimetic peptides. 8-well glass-bottom chamber slides (Nunc™ Lab-Tek™, Thermo Scientific, USA) were coated with phosphate-buffered saline (PBS) (control group, CTR), single (GER or GVM1) or combined (GER+GVM1) peptides (25 μg/mL each). After overnight-incubation at 4 °C, the slides were washed with PBS before cell seeding. Cells (MC3T3E1 cells, subclone 14) were seeded on the PBS-coated or peptide-coated slides (30K cells/well) and incubated in serum-free medium with 0.01% bovine serum albumin (BSA). After an 8-hour incubation, cells were washed with PBS, fixed with 4% PFA for 15 minutes at room temperature and incubated with rabbit polyclonal Phospho-FAK (Tyr397) antibody (1:200 dilution, 44-624, ThermoFisher) in PBSA (0.1% BSA in PBS) buffer overnight at 4 °C. Cells were incubated with Goat anti-rabbit secondary antibody (ALEXA FLUOR™ 555, ThermoFisher, A-21429, 1:500 in PBS) for 60 minutes at room temperature. Then, a second block with 10% normal rabbit serum in PBSA was performed for 30 minutes at room temperature followed by a 30-minute washing in PBS and the third block with unconjugated goat anti-rabbit Fab-fragment (dilution to 30 μg/mL in PBSA, 111-007-003, Jackson ImmunoResearch, USA) for 1 hour at room temperature. Afterwards, cells were incubated with Phospho-DDR2 antibody (1:200 in PBSA, MAB25382, R&D systems, USA) at 4 °C overnight. After washing with PBST (0.1% TWEEN@20 detergent in PBS), cells were incubated with a Goat-anti-rabbit secondary antibody (1:500 dilution in PBS, FITC, F2765, ThermoFisher, USA). Subsequently, cells were incubated with Phalloidin-Fluor 647 reagent (1:1000 dilution in PBS with 1% BSA, ab176759, Abcam, USA) for 1 hour at room temperature. After washing with PBST, cells were mounted with prolong gold antifade reagent with 4′, 6-diamidino-2-phenylindole (DAPI, P36931, ThermoFisher, USA).

The stained cells were imaged (images are not reproduced herein) with a Nikon C2 Confocal Microscope. The fluorescence intensity in red and green channels was measured in 3 areas per well, 3 wells for each group. Image J was used to quantify the average cell fluorescence (CTCF/Cell Number), where CTCF (Total Cell Fluorescence)=Integrated Density (rho−r (g/cm\hat{ }2))−(Area of selected cell×Mean fluorescence of background readings). The quantified results from the IF images are shown in FIG. 7A and FIG. 7B for pFAK and pDDR2, respectively.

In agreement with the quantified Western blot results (FIG. 6B and FIG. 6C), the IF microscopy of MC3T3E1 cells plated on peptide-coated tissue culture plates also showed selective activities of GER and GVM peptides on pFAK or pDDR2, respectively, and significant enhancement of both signals with the combined peptides. Taken together, the results confirm that collagen-binding integrin and DDR-activating peptides can cooperatively interact to stimulate integrin and DDR2 signaling.

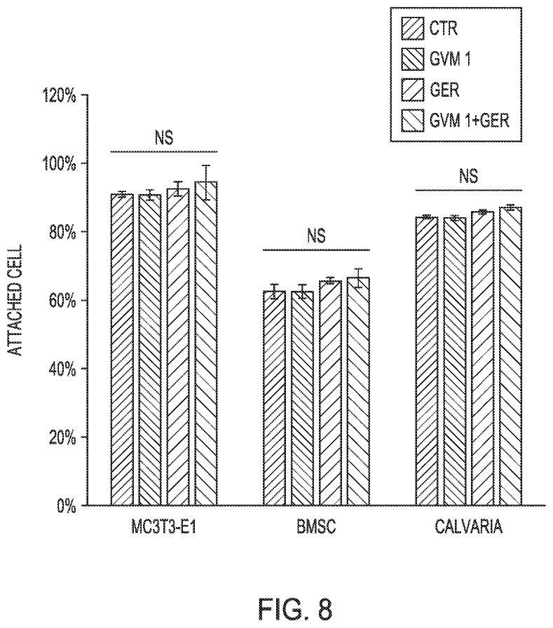

Moreover, these results cannot be explained by peptide-dependent differences in cell attachment that were uniformly high in the presence or absence of peptides (see FIG. 8). To assess cell attachment, MC3T3-E1cl14, BMSCs, and primary calvarial osteoblasts were plated on tissue culture plates with or without the indicated peptide coatings (25 μg/mL of GVM1, GER, or GVM1+GVR), and the percentage of cells attached was determined after 8 hours.

To measure the effects of the GVM1 peptide on osteoblast differentiation, the MC3T3E1cl14 preosteoblast cells were seeded on uncoated or GVM1 peptide-coated tissue culture dishes (25 μg/mL) and grown in osteogenic medium for up to 2 weeks. Alizarin red staining was used to detect mineralization, and Ddr2 and osteoblast marker (Alpl and Bglap) mRNAs were measured by qRT-PCR.

More specifically, to measure mineralization, the cells were fixed in 4% paraformaldehyde (PFA) for 15 minutes at room temperature and incubated with 40 mM Alizarin Red S solution (pH 4.2) for 2 hours at room temperature. After removal of the Alizarin Red S solution, the cells were washed with H₂O 5 times. Images were taken using a scanner. While the images are not reproduced herein, these results visually illustrated that growth on the GVM1 peptide enhanced mineralization compared to the control example.

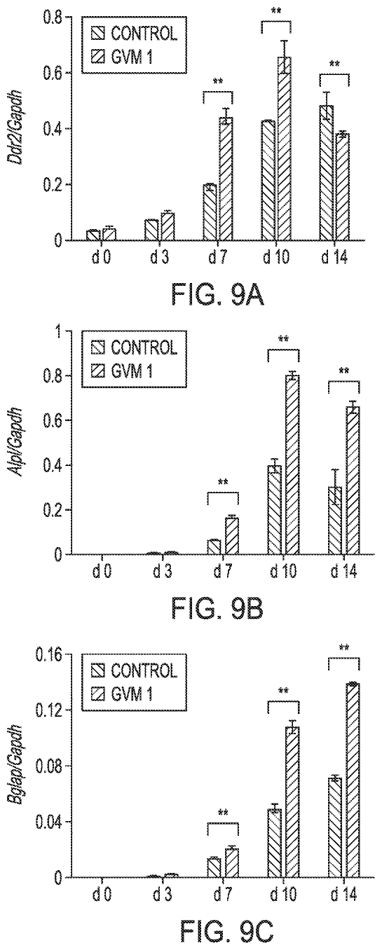

For gene expression analysis, the cells were harvested in TRIzol reagent (Invitrogen). Total RNA was prepared and reverse-transcribed to cDNA using Taqman reverse transcription kit (Applied Biosystem, N808-0234). Quantitative real-time PCR (qRT-PCR) was performed on an ABI PRISM 7700 sequence detector (Applied Biosystems). The following Taqman probes (Applied Biosystems) were used: Discoidin domain receptor 1 (Ddr1, Mm01273496), Discoidin domain receptor 2 (Ddr2, Mm01281887), von Willebrand Factor (Vwf, Mm00550376), Secreted Protein Acidic and Rich in Cysteine (Sparc, Mm05915229), Alkaline phosphatase (Alpl, Mm00475834), Osteocalcin (βglap, Mm03413826), Bone sialoprotein (Ibsp, Mm01208378) and Gapdh (Mm99999915). Levels of mRNAs were normalized with Gapdh. All real-time PCR results were calculated from triplicate RNA samples. As illustrated in FIG. 9A, Ddr2 expression increased during osteoblast differentiation, but was further increased in the presence of GVM1 peptide. Growth on the GVM1 peptide also stimulated the expression of the osteoblast differentiation marker mRNAs, alkaline phosphatase (Alpl) (FIG. 9B) and osteocalcin (Bglap) (FIG. 9C).

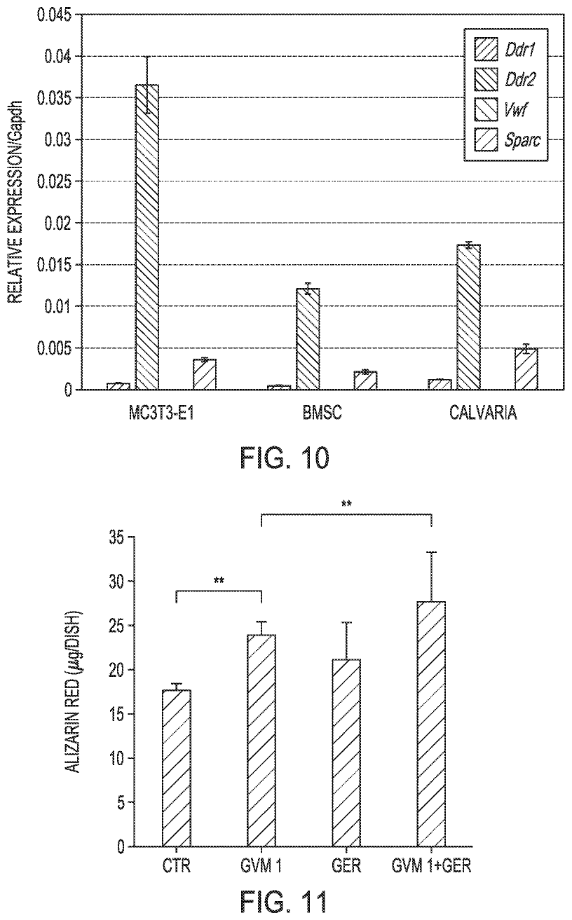

As mentioned herein, the GVM1 peptide can interact with other collagen-binding proteins in addition to DDR2, such as DDR1, vWF, and SPARC. To assess the potential contribution of these proteins to GVM1 peptide responsiveness of bone-derived cells, their expression was compared with DDR2 in MC3T3-E1, BMSCs, and COB cells. Total RNA was isolated and mRNA levels were determined by qRT-PCR. The results are shown in FIG. 10. In all 3 cell types, DDR2 was the predominant mRNA species with low amounts of DDR1 and SPARC mRNA also present. DDR1 mRNA levels were 2.5, 3.5 and 7 percent of DDR2 mRNA levels in MC3T3E1, BMSC and COB cells, respectively, while levels of SPARC mRNA were somewhat higher (9, 15 and 28 percent of DDR2 mRNA). vWF mRNA was not detected. Based on these results, it is believed that most of the response of bone cells to the GVM1 peptide is mediated by DDR2, SPARC and DDR1 may also be involved.

To evaluate activities of GVM1 and GER peptides, individually and cooperatively, on osteoblast differentiation, MC3T3E1cl14 cells were plated on tissue culture plates coated with the GVM1 peptide (25 g/mL), the GER peptide (25 μg/mL) or both peptides (25 μg/mL each). Cells were then grown in osteogenic medium for up to 2 weeks.

Alizarin red staining as described herein was used to detect mineralization. For these samples, the results were quantified. For quantification, 10% acetic acid was added to wells at room temperature for 30 minutes. Cell layers were collected and heated at 85 °C for 10 minutes. 10% ammonium hydroxide was used to neutralize acetic acid. The absorbance at 405 nm was measured and compared with Alizarin Red S standards. These results are shown in FIG. 11. As depicted, the GVM1 peptide significantly increased mineralization after 2 weeks while the GER peptide, by itself, did not have as significant of activity. However, the combination of the GER and GVM1 peptides significantly increased mineralization above levels seen with GVM peptide alone.

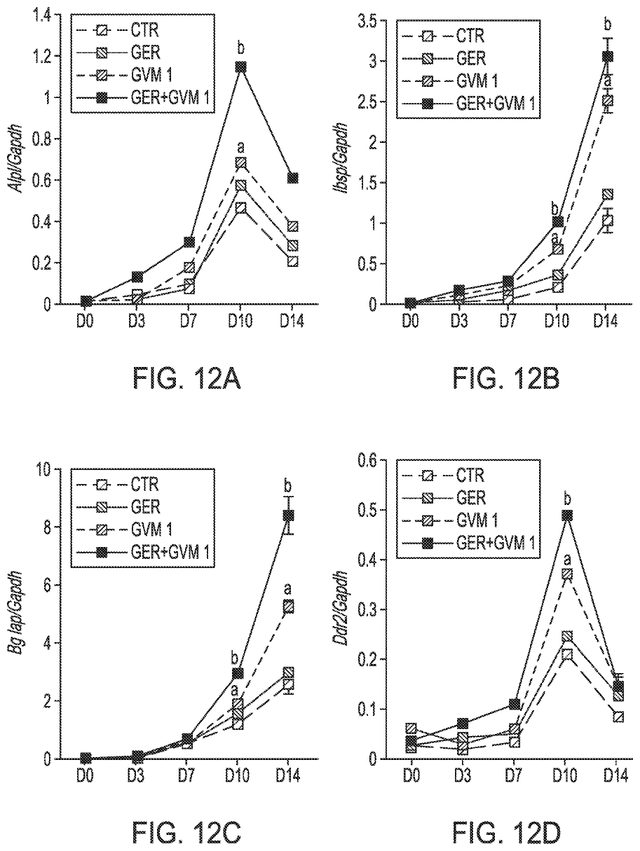

Gene expression analysis was also performed as described herein, where total RNA was collected at the times indicated in FIG. 12A through FIG. 12D, and qRT-PCR was used to measure the following mRNAs: Alpl, Ibsp, Bglap and Ddr2. When compared with control cultures, the GER peptide did not significantly increase mRNA expression at any of the times examined. In contrast, the GVM1 peptide increased Alpl, Bglap and Ibsp mRNAs at the 10- and 14-day time points, and increased Ddr2 mRNA at day 10. Consistent with the mineralization results shown in FIG. 11, the combination of both GVM and GER peptides further stimulated Alpl, Bglap and Ibsp mRNA expression beyond levels seen with the GVM1 peptide alone at day 10 and 14 and Ddr2 mRNA at day 10.

The MC3T3E1cl14 cells used in Example 1 are a subclonal cell line that was initially selected for robust mineralization capacity when grown in standard osteogenic media. This innately strong mineralization capacity may lead to the underestimation of effects of integrin and DDR-activating peptides. To measure the activity of peptides in progenitor cells having a lower intrinsic osteoblast differentiation potential, primary cultures of murine bone marrow stromal cells (BMSCs) were used. The BMSC cell population is known to contain skeletal stem cells and has been used extensively in bone regeneration applications. BMSCs were plated on peptide coated tissue culture plates and grown in osteogenic medium for 3 weeks before analysis.

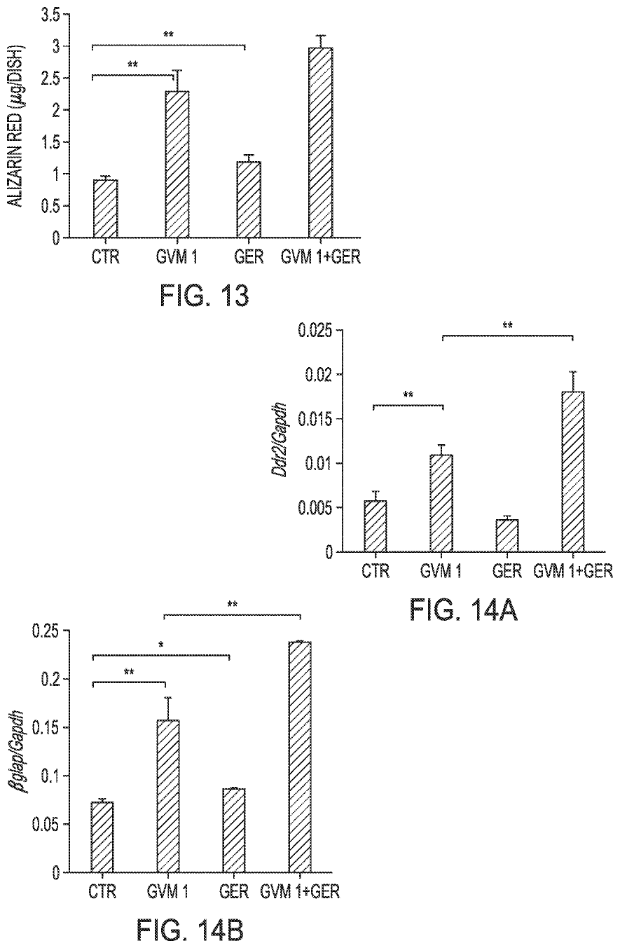

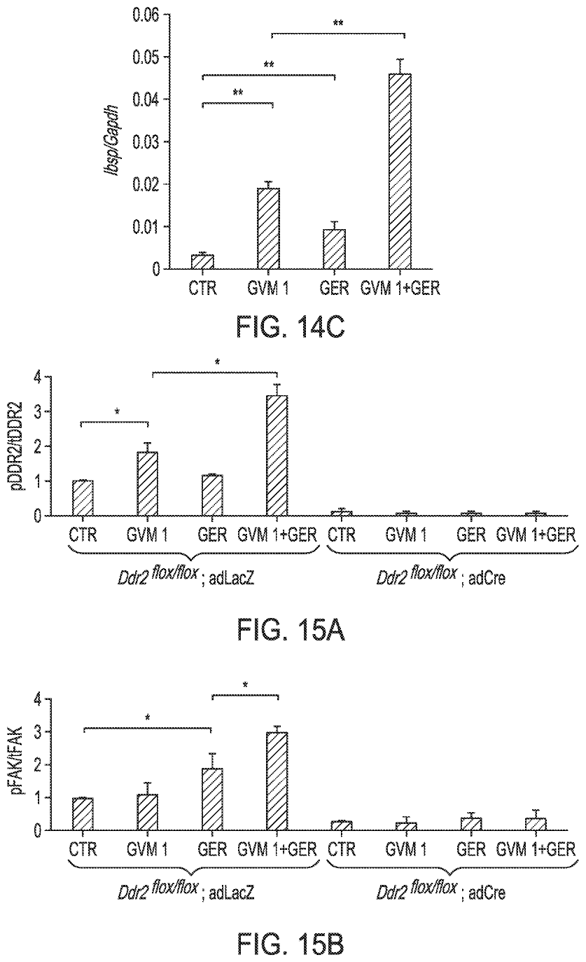

Both Alizarin red staining and gene expression analysis were performed as described herein. The quantified Alizarin red staining results are shown in FIG. 13, and the qRT-PCR measurements for the following mRNAs: Ddr2, Bglap, and Ibsp are shown in FIG. 14A, FIG. 14B, and FIG. 14C, respectively.

The GVM peptide strongly stimulated mineralization and expression of Ddr2, Bglap and Ibsp mRNAs when compared with control cultures, while the GER peptide was only slightly stimulatory for Bglap and Ibsp mRNAs, but did not significantly affect Ddr2 mRNA. Plating cells on both peptides further stimulated mineral and osteoblast marker expression to a greater extent than was seen with either peptide alone. For mineralization (FIG. 13), the stimulatory effects of peptides were approximately additive (a 3.4-fold stimulation for combined peptides vs. 2.5 and 1.3-fold stimulations for GVM and GER peptides individually) while mRNA inductions were additive to synergistic (FIG. 14A through FIG. 14C). For Bglap mRNA, a 3.3-fold stimulation was seen for combined peptides vs. 2.2- and 1.2-fold stimulation for the GVM and GER peptides individually. For Ddr2 mRNA, a 3.2-fold stimulation was obtained for combined peptides vs. 1.9- and 0.6-fold stimulations for GVM and GER peptides individually. For Ibsp mRNA, a 14.6-fold stimulation was seen for combined peptides versus 6 and 2.8-fold stimulations for GVM and GER peptides individually.

A gene inactivation approach was used to assess the requirement for Ddr2 in the peptide response. For these experiments, tissue culture dishes were coated with 25 μg/ml each of GVM1, GER, or both peptides. Primary calvarial preosteoblasts (COB cells) were isolated from Ddr2^{flox/flox} mice and transduced with a control adenovirus (adLacZ) or Cre-expressing adenovirus (adCre) to inactivate Ddr2 by homologous recombination. COB cells are enriched in calvarial suture stem cells as well as periosteal cells and are a well-established model for studying osteoblast differentiation. The adenovirus-treated COB cells were seeded on the tissue culture plates coated with GVM1, GER or both peptides.

Primary calvaria osteoblast cells (COBs) isolated from Ddr2^{flox/flox} mice were treated with adLacZ (control) or adCre and plated on peptide coated dishes. DDR2 and FAK phosphorylation were then measured after 8 hours. More particularly, DDR2 activation (pDDR2/tDDR2) and FAK activation (pFAK/tFAK) were measured by Western Blot (results not reproduced herein) and quantified by densitometry. The DDR2/tDDR2 and pFAK/tFAK results are respectively shown in FIG. 15A and FIG. 15B. Like the results obtained with MC3T3E1 cells (FIG. 6B and FIG. 6C), in control COB cells (adLacZ-transduced), DDR2 activating peptide (GVM1) increased pDDR2/tDDR2 1.8-fold without affecting pFAK/tFAK while integrin-activating peptide (GER) stimulated pFAK/tFAK 1.9-fold without affecting DDR2 phosphorylation. The combination of both peptides increased both pDDR2/tDDR2 and pFAK/tFAK to a greater extent than was seen with either individual peptide (3.4-fold and 3.0-fold stimulation versus control for pDDR2/tDDR2 and pFAK/tFAK, respectively). Significantly, in adenoCre-treated cells, not only was DDR2 protein reduced to undetectable levels, but also the stimulation of FAK phosphorylation by GER peptide with or without GVM1 peptide was abolished without changing total FAK levels. The results in FIG. 15A and FIG. 15B thus illustrate that the DDR2 and integrin activating peptides synergistically stimulating osteoblast differentiation in a DDR2-dependent manner.

To measure the requirement for Ddr2 on osteoblast differentiation in the presence of peptides, control (adLacZ-treated) or adCre-treated COB cells were seeded on the peptide-coated dishes and grown in osteogenic medium for 3 weeks before measurement of mineralization and osteoblast marker mRNA expression.

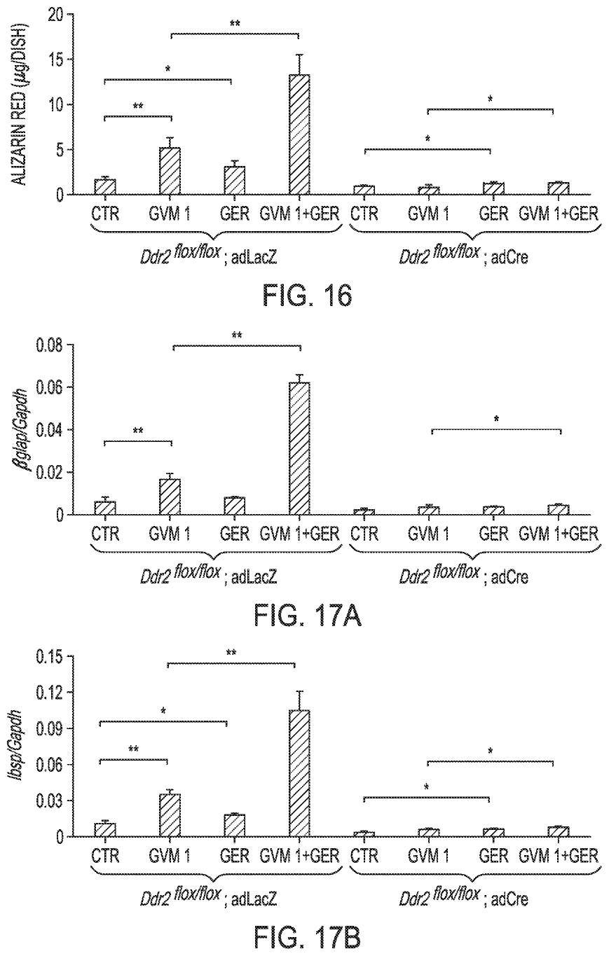

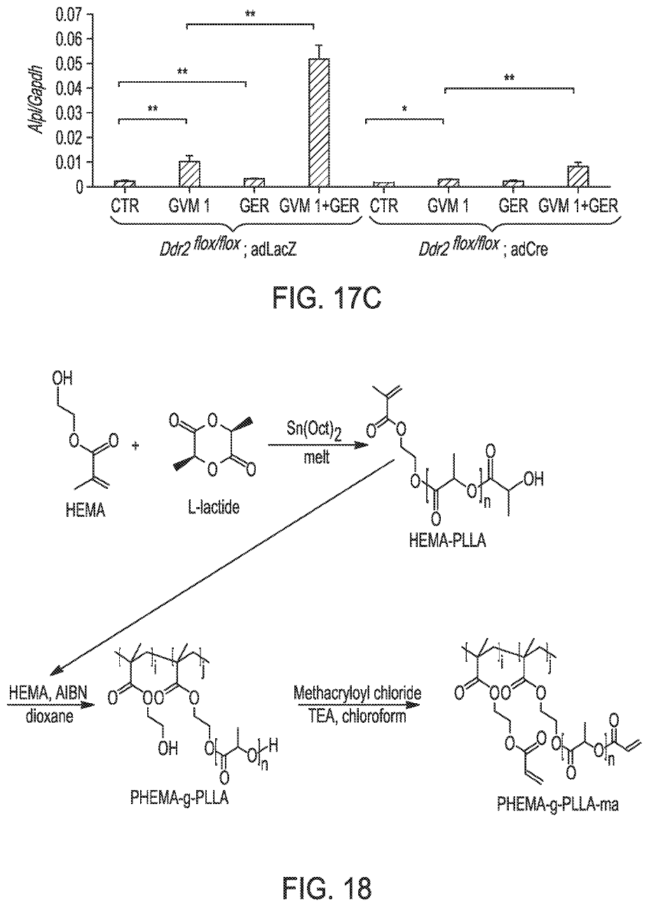

Both Alizarin red staining and gene expression analysis were performed as described herein. The quantified Alizarin red staining results are shown in FIG. 16, and the qRT-PCR measurements for the following mRNAs: Bglap Ibsp, and Alpl are shown in FIG. 17A, FIG. 17B, and FIG. 17C, respectively.

As shown in FIG. 16, and FIG. 17A through FIG. 17C, the GVM1 peptides and, to a lesser extent, the GER peptides stimulated osteoblast differentiation in control COB cells (adLacZ samples) with combined peptide treatment (GVM1+GER) being synergistic relative to the individual peptides.

As depicted in FIG. 16 for the control COB cells (adLacZ samples), the GVM1 peptide treatment increased mineralization 3.1-fold, the GER peptide increased mineralization 1.9-fold, while the combined peptide treatment increased mineralization 8.3-fold. In contrast, peptide effects on osteoblast mineralization were almost completely abolished with Ddr2 inactivation (adCre samples).

As shown in FIG. 17A through FIG. 17C, synergistic effects of peptides on control COB cells were also observed when osteoblast marker mRNAs (adLacZ samples) were measured. For Bglap mRNA (FIG. 17A), a 10.7-fold induction was seen with the combined peptides vs. 2.7 and 1.3-fold induction for the GVM1 and GER peptides individually. For Ibsp mRNA (FIG. 17B), a 10.3-fold induction was obtained with the combined peptides versus 3.3 and 1.7-fold stimulations for the GVM1 and GER peptides individually. For Alpl mRNA (FIG. 17C), a 20.7-fold stimulation was seen for the combined peptides vs. 4.0 and 1.3-fold stimulations for the GVM1 and GER peptides individually. In contrast, mRNA induction by peptides in adCre treated cells (control) was nearly eliminated.

The Western blot, mineralization and gene expression analyses showed DDR2 to be necessary not only for cellular responsiveness to the DDR activating peptide, but also for the response to the integrin activating peptide (compare the response to GVM1 and GER peptides in adLacZ versus adCre-treated cells). This suggests that DDR2 is necessary for full β1 integrin activity.

### Example 2

In this example, the following materials were used: Poly(L-lactide) (PLLA, RESOMER® L 207 S), L-lactide (LA), azobisisobutyronitrile (AIBN), 1,4-dioxane, methanol, dichloromethane (DCM), triethylamine (TEA), methacryloyl chloride, tetrahydrofuran (THF), ethanol, mineral oil, sodium hydrogen carbonate (NaHCO₃), Tin(II) 2-ethylhexanoate (Sn(Oct)₂), hydroxyethyl-methacrylate (HEMA), hydrochloric acid (HCl), fructose, phosphate buffered saline (PBS), and sodium chloride (NaCl).

### Polymer Synthesis

FIG. 18 depicts the reaction scheme for the HEMA-PLLA Macromonomer, Poly(HEMA-graft-PLLA), and the poly(HEMA-graft-PLLA)-methacrylate.

### Synthesis of HEMA-PLLA Macromonomer

At a fixed mole ratio, L-lactide and HEMA were added into a 100 ml round-bottomed flask. Sn(Oct)₂ with 0.01 mol % of L-lactide was added as the catalyst. The flask was sealed and nitrogen gas was used to purge the container for 10 minutes. The container was put onto a glycerol bath at 140 °C for about 2 hours. The reaction mixture turned from a transparent liquid to a white, opaque, viscous liquid. The container was then cooled down, in air, to room temperature. Dichloromethane was added to the reaction mixture to make the weight percentage of L-lactide to be 20%. The precipitate caused by the remaining Sn(Oct)₂ was removed by centrifuge. The polymer/dichloromethane solution was pulled into cold methanol (1:10 volume ratio) and immediately centrifuged. The white precipitate was washed with methanol two more times and MILLI-Q® water three times, and then was freeze-dried to form a white solid product.

For this macromonomer, typical reagent amounts included 11.53 g L-lactide, 0.928 g HEMA and 0.324 g Sn(Oct)₂, and about 9.4 g of the product was collected.

### Synthesis of Poly(HEMA-graft-PLLA) (PHEMA-q-PLLA)

The HEMA-PLLA macromonomer was dissolved in 1,4-dioxane before it was added into a 100 ml round-bottomed flask. To achieve complete dissolution, 10 wt % of the HEMA-PLLA macromonomer was maintained in 1,4-dioxane at 60 °C for about 12 hours. A fixed mole ratio of HEMA and AIBN was then added into the solution, and nitrogen gas was used to purge the container for 10 minutes. The container was put onto a glycerol batch at 80 °C for 24 hours. The reaction mixture was transparent. The transparent reaction mixture was then pulled into cold methanol (1:10 volume ratio) and centrifuged at 1,500 g for 3 minutes. The white flocculent precipitate was then washed with methanol two more times and MILLI-Q® water three times, and then freeze-dried to form white solid product.

For poly(HEMA-graft-PLLA), typical reagent amounts included 2.7 g HEMA-PLLA macromonomer (MW 3,000), 1.17 g HEMA and 29.4 mg AIBN, and about 2.4 g of the product was collected.

### Synthesis of poly(HEMA-graft-PLLA)-methacrylate (PHEMA-q-PLLA-ma)

5 wt % of the PHEMA-g-PLLA was dissolved in dichloromethane in a 100 ml round-bottomed flask. The flask was sealed and purged with nitrogen gas for 5 minutes, and then it was placed in an ice-water bath for at least 10 minutes before the reaction was initiated. Fixed mass ratios of TEA:PHEMA-g-PLLA (0.6:1) and methacryloyl chloride:PHEMA-g-PLLA (0.4:1) were pre-dissolved in a small amount of DCM. The TEA solution was added into the flask first, and the methacryloyl chloride solution was added into the flask drop-wise to prevent rapid increase of temperature. The flask was kept in the ice-water bath for at least 1 hour. The resulting liquid mixture went from a red color to an orange color, and then was washed with 50 ml 0.1 M HCl two times, or until pH of the water phase became lower than 7, and then was washed with 50 ml 0.5M NaHCO₃ one time. The organic phase was then pulled into cold methanol (1:10 volume ratio) and was immediately centrifuged briefly to form a white precipitate. The precipitate was washed with methanol two more times and MILLI-Q® water three times, and then was freeze-dried to form a white solid product.

For PHEMA-g-PLLA-ma, typical reagent amounts included 2.5 g PHEMA-g-PLLA (M.W. 20k), 1.0 g methacryloyl chloride, 1.5 g TEA, and about 1.8 g product is collected.

### Polymer Characterization

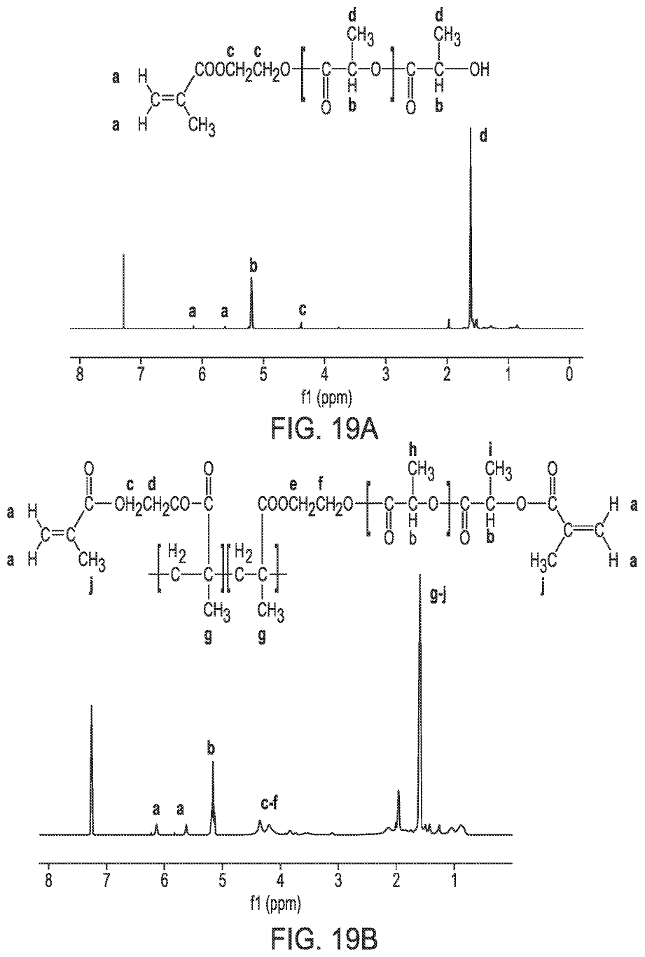

The chemical structure of PHEMA-g-PLLA-ma was verified by H NMR (see FIGS. 19A and 19B). FIG. 19A depicts the H NMR spectrum of the HEMA-PLLA macromonomer, and FIG. 19B depicts the H NMR spectrum of PHEMA-g-PLLA-ma. The following peaks were used to analyze the ratio of blocks. The peak at δ=6.14 and δ=5.62 represented H on methacrylate functional groups. Peaks at δ=4.35 and 4.19 were assigned to —OCH₂OCH₂— units. The quartet at δ=5.16 was assigned to CH in the lactic acid unit. The length of the PLLA fragment was obtained by the H NMR result for HEMA-PLLA, which was 40.7 units per chain. The ratio of HEMA and PLLA in PHEMA-g-PLLA-ma was calculated to be 8.9, and methacrylate density was 6.2 per PLLA chain. GPC gave MW of 21,000 for PHEMA-g-PLLA-ma.

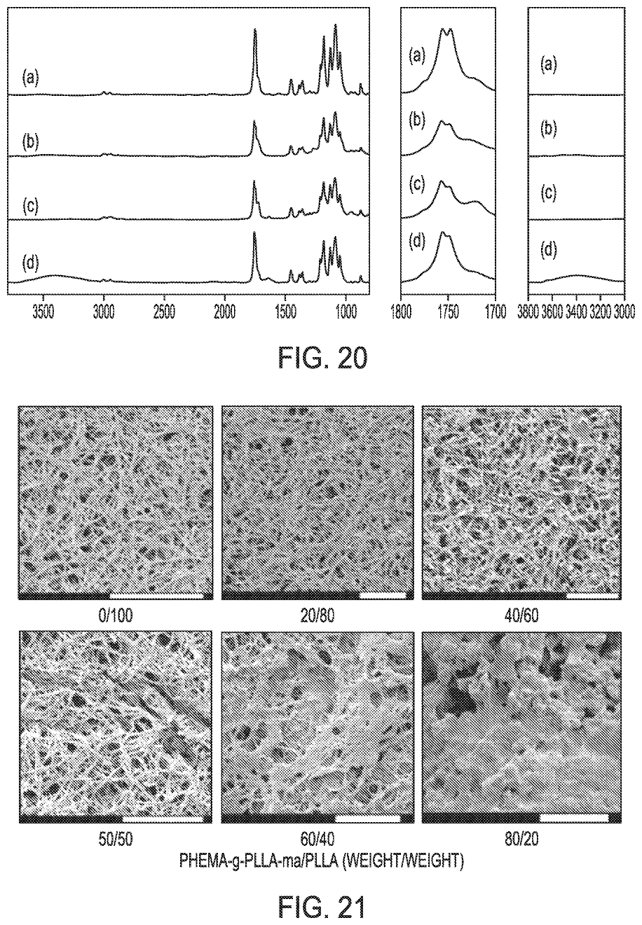

The chemical structure of PHEMA-g-PLLA-ma was also verified by FTIR (see FIG. 20, see (a), (b), and (c)). The reaction of peptides on the surface of the nanofibrous structure (described below) was also qualified by FTIR (see FIG. 20, (d)). The peak at 1724 cm⁻¹ representing C═O conjugated with C═C increased with the introduction of the methacrylic group from PHEMA-g-PLLA to PHEMA-g-PLLA-ma, and decreased after reaction with the peptide. The appearance of the broad peak at 3380 cm⁻¹ also suggested the presence of attached peptides.

Nanofibrous Film Fabrication with PHEMA-g-PLLA-Ma/PLLA Blend

Nanofibrous films were prepared using a thermal induced phase separation method. 10 wt % of PHEMA-g-PLLA-ma was dissolved in tetrahydrofuran and 10 wt % PLLA was dissolved in tetrahydrofuran at 60 °C until the solutions became transparent. Then, the two solutions were mixed together based on the desired blend ratio. The viscous polymer solution was then poured into a mold with a silicon surface (bottom) and glass surface (top) separated with 4 strips of adhesive tape as spacers to control the height of the films. After adding the polymer, the mold was immediately sealed and placed into a −80 °C freezer for 2 days to induce phase separation. The mold was then submerged in an ice-water batch while it returned to room temperature. The solidified film was then completely dried on a bench top and stored in vacuum until further experiments.

A variety of phase separation conditions were examined to ensure the nanofiber morphology was maintained. Films were made from polymer blends containing different weight ratios of PHEMA-g-PLLA-ma:PLLA (0:100, 20:80, 40:60, 50:50, 60:40, and 80:20), and were observed under scanning electron microscopy (SEM). The results are shown in FIG. 21 and illustrate that when the weight percentage of PHEMA-g-PLLA-ma in the polymer blend was higher than 50%, the nanofibrous structure of the film largely disappeared. Thus, it is desirable to use 50% or less of the PHEMA-g-PLLA-ma in a blend with PLLA or another like polymer (e.g., PLGA, etc.).

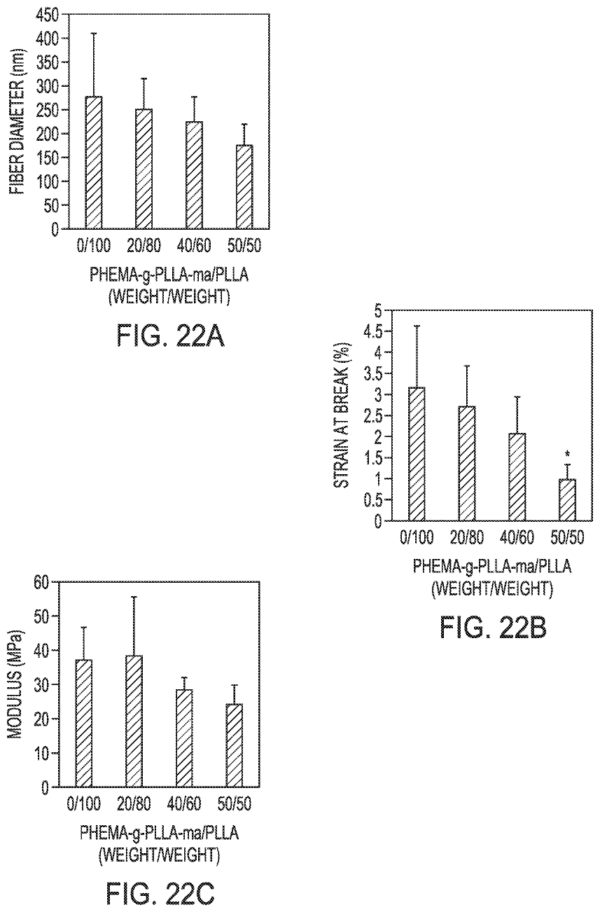

The SEM images were analyzed for fiber diameter, and these results are shown in FIG. 22A. FIG. 22A illustrates that there was no statistically significant difference in fiber diameter among the nanofibrous films with different PHEMA-g-PLLA-ma/PLLA blend ratios, but a trend of decreasing diameter was observed with decreasing PLLA content in the polymer blend.

Tensile test on the films were performed. In particular, the tests included strain at break (FIG. 22B) and Young's Modulus (FIG. 22C). FIG. 22B shows that the strain at break decreased with decreasing PLLA content in the polymer blend. There was no statistically significant difference in modulus among the samples with different PHEMA-g-PLLA-ma/PLLA blend ratios, although there was a trend of decreasing modulus with decreasing PLLA content (FIG. 22C).

Nanofibrous 3D Scaffold Fabrication with PHEMA-g-PLLA-Ma/PLLA Blend

Fructose templates were formed by cooling down a fructose/mineral oil emulsion from 120 °C to an ice-water bath and collecting fructose spheres with diameter of 250 μm to 425 μm using stainless steel sieves. The fructose spheres were immersed in hexane and annealed into a connected template in TEFLON® vials at 37 °C. The templates were then vacuum dried to remove hexane. 10 wt % of the respective PHEMA-g-PLLA-ma/PLLA blends were dissolved in THF at 60 °C until the solutions became transparent. Each viscous solution was then pulled into a respective TEFLON® vial. Brief vacuum was used to remove air from the fructose template, and to allow the polymer solution to fill in the pores of the template. Each vial was then sealed and placed in a −80 °C freezer for 2 days to induce phase separation. The vials, each with an opaque gel-like polymer filling the fructose template, were then unsealed, soaked in hexane to exchange with THF, and then soaked in water to remove the fructose templates. The porous 3D nanofibrous scaffolds were collected, cut into desired shapes, and freeze dried for subsequent experiments.

### Peptide Conjugation on PHEMA-g-PLLA-Ma/PLLA Blend Nanofibrous Structure

### Quantification of Peptide Conjugation

A DDR2 binding peptide (GVM2) and two integrin binding peptides (GER2 and GER3) were synthesized by Alan Scientific Inc. As depicted, all the sequences were flanked by GPP on both sides which enabled the triple helix structure of peptides in aqueous solution.

GVM2 had the following sequence:

GPC(GPP)₅GPRGQOGV(Nle)GFO(GPP)₅GGP-NH₂ (SEQ. ID. NO. 5 + -NH₂)

and GER2 and GER3 had the following sequences, respectively:

GGP(GPP)₅GFOGER(GPP)₅GPC-NH₂ (SEQ. ID. NO. 6 + -NH₂).
GGP(GPP)₅GFOGER(GPP)₅GPC-NH₂-biotin (SEQ. ID. NO. 6 + -NH₂-biotin).

For Example 2, the peptides were conjugated to the surfaces of the nanofibrous films or the 3D scaffolds as described in Example 3. Additionally, peptide conjugation was examined as described in Example 3.

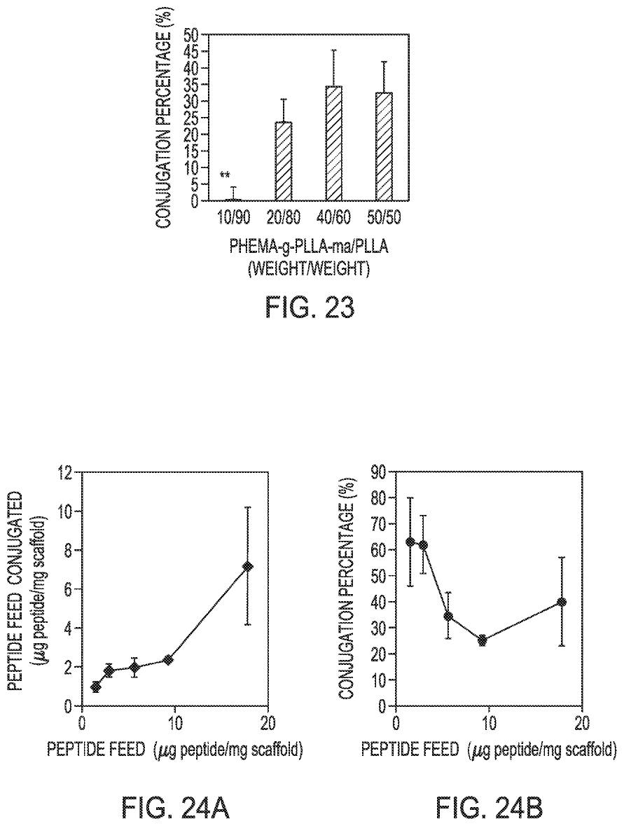

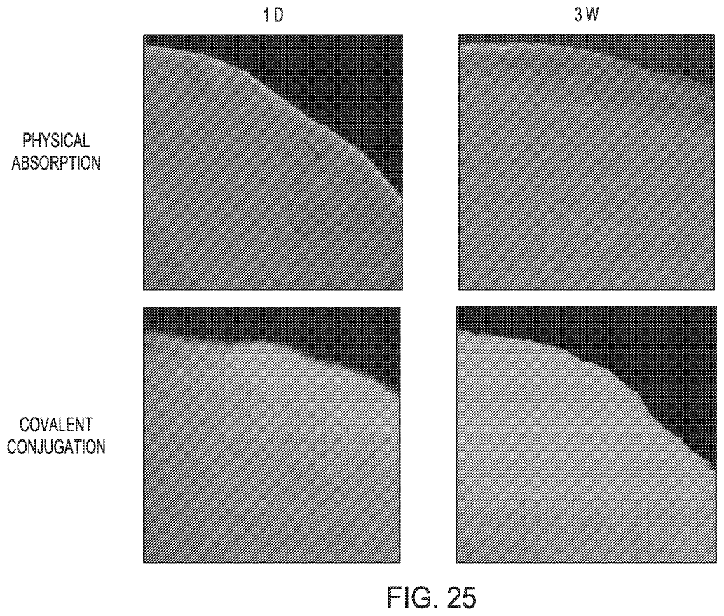

The percentage of the peptide conjugated on the films with different blend ratios is shown in FIG. 23. As depicted, there was an increasing trend as the portion of PHEMA-g-PLLA-ma increased. At a blend ratio of 10 wt % (PHEMA=10 wt %), there was almost no observed peptide conjugation. It is believed that these results are due to limited methacrylate groups exposed on the fiber surface. As such, the weight ratio of PHEMA-g-PLLA-ma to PLLA should be greater than 10:90.

It was observed that different amounts of peptide were attached to the 3D nanofibrous scaffolds with a fixed PHEMA-g-PLLA-ma:PLLA blend ratio of 50/50 when different amounts of peptide were used. These results are shown in FIG. 24A and FIG. 24B. FIG. 24A depicts the conjugated peptide per mass of 3D scaffold (μg/mg) before and after conjugation. As depicted in FIG. 24A, the peptide conjugation increased as peptide feed increased. FIG. 24B depicts the conjugation percentage changing with feeding percentage. As depicted in FIG. 24B, at higher peptide feed amounts, the conjugation percentage exhibited a decreasing trend.

### Visualization of Peptide Conjugation

Two different labeling techniques were used to visually analyze the peptide conjugation. The first method involved the GFO-biotin peptides (i.e., GER3, see SEQ. ID. NO. 6+-NH₂-biotin). In the first method, a streptavidin-ALEXA® Fluor 555 conjugate (referred to as “Avidin-Alex”) was used to label the peptide. In the second method, Fluorescein isothiocyanate isomer I (FITC dye) was used to label the amino terminal of the GVM2 peptides (see SEQ. ID. NO. 5+−NH₂) in pH 9.0 phosphate buffer, followed by dialysis purification. A Leica Thunder microscope was used to observe the two dyes at corresponding wavelengths. [0160]10 μg of the labeled GER3 was conjugated to the nanofibrous films (5 mm diameter). For covalent conjugation, the nanofibrous films were soaked in 10 μg GFO-biotin/200 μL TEA/MILLI-Q® water (pH=10) for 1 hour. [0161]10 μg of labeled GER3 was also physically absorbed to each nanofibrous film (5 mm diameter). For absorption, the nanofibrous films were soaked in 10 μg GFO-biotin/200 μL PBS at 4 °C overnight.

All of the films were then placed in PBS solution at 37 °C for up to 3 weeks. The amount of remaining peptide was visualized by the strength of fluorescence after staining at different time periods (e.g., 1 day, 3 week). The fluorescent images, which are reproduced herein in black and white, are shown in FIG. 19. After 3 weeks, the remaining peptide content on the physically adsorbed samples decreased, while the remaining peptide content on the conjugated samples was almost unchanged. The results suggested firm conjugation of the peptides was achieved.

### Cell Distribution, Viability in Nanofibrous Scaffolds

Ddr2 signaling regulates osteogenic and odontoblastic differentiation potential of the multi-potent periodontal ligament (PDL) cells (PDLCs). In this example, these multipotent stem/progenitor cells were used to evaluate their seeding, distribution, and viability/proliferation in nanofibrous PLLA-g-HEMA scaffolds with or without GER2/GVM2 peptides. When used, the unlabeled peptides were covalently conjugated following the same procedure described herein. The nanofibrous PLLA-g-HEMA scaffolds with or without GER2/GVM2 peptides were incubated in the cells, and test were performed after overnight incubation and then again after 7 days of incubation.

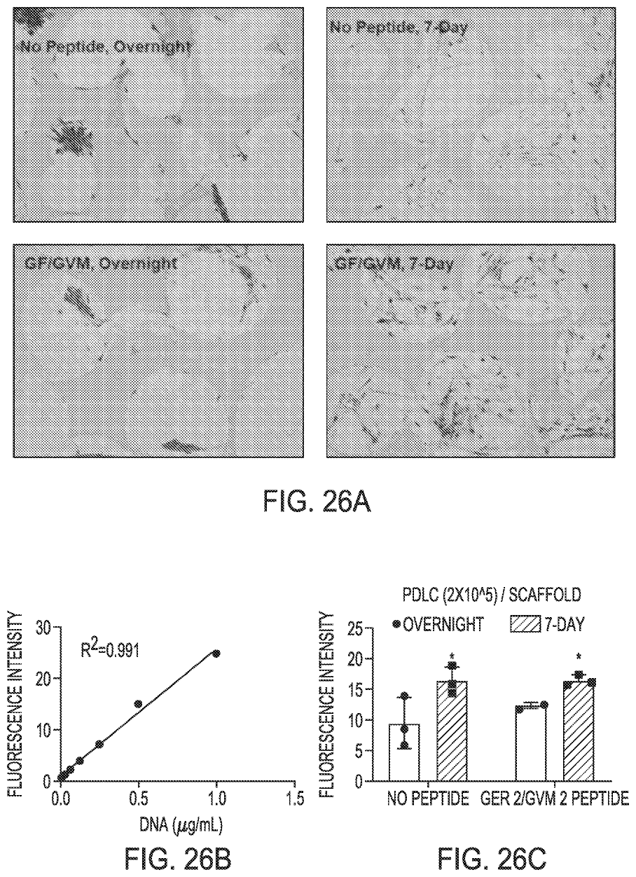

H&E staining was performed after both incubation periods. The images are shown in FIG. 26A. These images demonstrate quite uniform cell seeding/distribution in both types of scaffolds (without and without peptides). As depicted, however, there was a significant increase of cell numbers with more cell matrix after 7-day incubation versus overnight incubation. These results were consistent with the DNA counting data shown in FIG. 26B and FIG. 26C.

### Example 3

In this example, the following materials were used: Poly(L-lactide) (referred to as pure PLLA, RESOMER® L 207 S, Mn=176k), L-lactide (La), azobisisobutyronitrile (AIBN), 1,4-dioxane, methanol, dichloromethane (DCM), triethylamine (TEA), methacryloyl chloride, tetrahydrofuran (THF), ethanol, mineral oil, sodium hydrogen carbonate (NaHCO₃), anhydrous sodium sulfate, Tin(II) 2-ethylhexanoate (Sn(Oct)₂), hydroxyethyl-methacrylate (HEMA), triazabicyclodecene (TBD), mineral oil, SPAN 80®, anhydrous sodium sulfate, IRGACURE® 2959, hydrochloric acid (HCl), fructose, phosphate buffered saline (PBS), sodium chloride (NaCl), saline, ethylenediaminetetraacetic acid (EDTA) and tromethamine (Tris). Custom peptides (SEQ. ID. NOS. 6-8) were purchased from Alan Scientific (US). C57BL/6J mice (female) of 6-to-8-week-old were purchased from The Jackson Laboratory.

### Polymer Synthesis

HEMA-PLLA macromonomers, poly(HEMA-graft-PLLA) copolymers, and poly(HEMA-graft-PLLA)-methacrylate copolymers were prepared in a similar manner as shown in FIG. 18.

HEMA-PLLA with different molecular weights was polymerized with L-lactide as the monomer and HEMA as the initiator. The HEMA-PLLA macromonomers with a molecular weight less than 20,000 g/mol were prepared as described in Example 2.

Any HEMA-PLLA macromonomer with a molecular weight greater than 20,000 g/mol was prepared as follows. TBD, L-lactide, and HEMA were pre-dried with anhydrous sodium sulfate prior to the reaction. A 20 ml vial with 1.5 g of molecular sieve was sealed and exposed to a nitrogen gas purge for 10 minutes. 10 wt % of L-lactide pre-dissolved in anhydrous DCM with HEMA of 1/100 to 1/800 mole ratio to L-lactide was added in the vial using a syringe. This is the ring-opening reaction using HEMA to initiate the PLLA chain polymerization. A syringe loaded with 2 mg/ml TBD solution (0.1 mol % of L-lactide) was set on the rubber cover with a TEFLON® filter. The device was cooled down at −80 °C for 1 hour before TBD solution was injected into the vial. The vial was set at −80 °C for 48 hours. The viscous reaction mixture was put on rotary evaporator to remove DCM. The product was washed off from the molecular sieve using an excess amount of DCM, and was vacuum dried to remove DCM. The monomer was washed away with ethyl acetate 2 times before the product was dried to form a transparent-to-opaque white film.

This macromer was mixed with HEMA to form (via radical polymerization) the graft copolymers PHEMA-g-PLLA in accordance with the process described in Example 2. The PHEMA-g-PLLA copolymers (see Table 2) were functionalized with methacrylate as described in Example 2. While the results are not shown herein, the change of double bond was confirmed by both NMR and FTIR for each of the PHEMA-g-PLLA-ma copolymers. The number average molecular weight of the resulting PHEMA-g-PLLA-ma copolymers was measured and is shown in Table 1.

For some applications, it is desirable for the biological compositions disclosed herein to have a nanofibrous architecture. PLLA is capable of generating nanofibrous architectures, but does not include peptide conjugating functional groups. As noted in Example 2, the graft copolymer, PHEMA-g-PLLA-ma, is capable of forming the nanofibrous architecture and incorporates the functionality of methacrylate in the repeating HEMA units and in the end groups for peptide conjugation by a thiol-ene click reaction. The ring-opening reaction to form the macromer (PLLA chain) is the process that determines the PLLA chain length in the graft copolymer. This process may result in shorter graft PLLA chain lengths, which can compromise the capacity for crystallization and nanofiber formation. To address this, blends of pure PLLA and different PHEMA-g-PLLA-ma graft copolymers were prepared and tested. There were three variables that controlled the composition of the PHEMA-g-PLLA-ma/PLLA blend: the blend ratio of PHEMA-g-PLLA-ma/PLLA, the length of PLLA chain in the PHEMA-g-PLLA-ma graft copolymer, and the ratio of HEMA/L-lactide used to generate the graft copolymer.

Table 1 identifies the PHEMA-g-PLLA-ma graft copolymer used to form various blends by its graft PLLA chain length, the HEMA-L-lactide ratio use in its formation, and its molecular weight. PLLA chain Mn and PDI and PHEMA-g-PLLA-ma Mn and PDI and were obtained by Gel Permeation Chromatography (THF solvent).

TABLE 1

| | PHEMA-g-PLLA-ma Graft Copolymer | | | |
|---|---|---|---|---|
| | Graft PLLA | HEMA/ | PHEMA-g- | Blend Ratio |
| | Chain | L-lactide | PLLA-ma | (PHEMA-g- |
| Blend | Mn/10\hat{ }³ | ratio | Mn/10\hat{ }³ | PLLA-ma/ |
| ID | (PDI) | (wt/wt) | (PDI) | PLLA) |
| A1 | 3.60 | 0.43 | 19.7 | 10/90 |
| A2 | (1.22) | | (1.25) | 20/80 |
| A3 | | | | 30/70 |
| A4 | | | | 40/60 |
| A5 | | | | 50/50 |
| B1 | 5.33 | 0.43 | 27.9 | 10/90 |
| B2 | (1.14) | | (1.42) | 20/80 |
| B3 | | | | 30/70 |
| B4 | | | | 40/60 |
| B5 | | | | 50/50 |
| C1 | 17.7 | 0.43 | 62.2 | 10/90 |
| C2 | (1.52) | | (1.56) | 20/80 |
| C3 | | | | 30/70 |
| C4 | | | | 40/60 |
| C5 | | | | 50/50 |
| C6 | 17.7 | 0.86 | 114 | 10/90 |
| C7 | (1.52) | | (1.19) | 20/80 |
| C8 | | | | 30/70 |
| C9 | | | | 40/60 |
| C10 | | | | 50/50 |
| D1 | 112 | 0.43 | 131 | 10/90 |
| D2 | (1.28) | | (1.26) | 20/80 |
| D3 | | | | 30/70 |
| D4 | | | | 40/60 |
| D5 | | | | 50/50 |
| D6 | 112 | 0.86 | 168 | 10/90 |
| D7 | (1.28) | | (1.41) | 20/80 |
| D8 | | | | 30/70 |
| D9 | | | | 40/60 |
| D10 | | | | 50/50 |
| E1 | 132 | 0.43 | 160 | 10/90 |
| E2 | (1.37) | | (1.55) | 20/80 |
| E3 | | | | 30/70 |
| E4 | | | | 40/60 |
| E5 | | | | 50/50 |
| E6 | 132 | 0.86 | 194 | 10/90 |
| E7 | (1.37) | | (1.28) | 20/80 |
| E8 | | | | 30/70 |
| E9 | | | | 40/60 |
| E10 | | | | 50/50 |
| F1 | 228 | 0.43 | 257 | 10/90 |
| F2 | (1.74) | | (1.29) | 20/80 |
| F3 | | | | 30/70 |
| F4 | | | | 40/60 |
| F5 | | | | 50/50 |
| F6 | 228 | 0.86 | 282 | 10/90 |
| F7 | (1.74) | | (1.43) | 20/80 |
| F8 | | | | 30/70 |
| F9 | | | | 40/60 |
| F10 | | | | 50/50 |

Higher blend ratios were prepared (PHEMA-g-PLLA-ma/PLLA of 60/40 and 80/20), but during film or scaffold formation (described below) these blends resulted in either non-fibrous morphology or loss of typical nanofibrous morphology (at least some of the fibers merged). While these films were not tested further in this Example, the non-fibrous morphology or merged fiber morphology may be useful for some applications of the biological composition.

Once the film or scaffold is generated, three important features of the polymeric structure include: nanofiber features (morphology and diameter), mechanical properties (e.g., modulus), and the achievable peptide conjugation density. These features are further described in reference to the films and/or scaffolds.

Nanofibrous Film Fabrication with PHEMA-g-PLLA-Ma/PLLA Blends

Nanofibrous films were prepared using a thermal induced phase separation method. 10 wt % of the polymer blends with various weight ratios of PHEMA-g-PLLA-ma and PLLA (as shown in Table 1) were respectively dissolved in tetrahydrofuran (THF) at 60 °C until the solutions became transparent. The viscous polymer solutions were then poured into respective preheated 0.5-mm-thick molds made of two pieces of glass slides with a silicon wafer as inner surface of the lower slide to ensure film flatness. The molds were immediately sealed and placed into a −80 °C freezer for 2 days to induce phase separation. The molds were then unsealed and submerged in an ice-water batch to exchange THF. The solidified films were then dried between paper towels on benchtop and stored in vacuum until further experiments.

Tensile modulus, strain at break, and ultimate strength were obtained from tensile tests for films with the following dimensions: 2-mm wide, 20-mm long and 0.2-mm thick (n=3). A GT-UA03 Single Column Tensile Test Machine with a 50 N loading cell was used for all mechanical tests. The samples were fixed on a small force film holder with a 10 mm gauge length and extended at a speed of 0.5 mm/min. Tensile modulus and strain at break were calculated from stress-strain curves.

Melting enthalpy of nanofibrous films was obtained using Differential Scanning Calorimetry (DSC). 5 mg to 10 mg of nanofibrous films were added to an aluminum sample holder and were heated from 50 °C to 190 °C at a speed of 20 °C per minute. Melting enthalpy was calculated using the built-in area calculation of the program.

Fiber diameters were measured using SEM images of some of the nanofibrous films (n=50-100). Films underwent gold coating and were observed under JEOL JSM-7800FLV SEM instrument. The images were analyzed, and average fiber diameter was calculated by measuring 50~100 fibers for each sample.

The results for each of these tests is shown in Table 2. The various films are identified by the blend used to form the film.

TABLE 2

| | Tensile | Strain at | Ultimate | Melt | Fiber |
|---|---|---|---|---|---|
| Blend | Modulus | Break | Strength | Enthalpy | Diameter |
| ID | (Mpa) | (%) | (Mpa) | (J/g) | (nm) |
| A1 | 114.3 ± 9.9 | 2.7 ± 0.8 | 2.4 ± 1.1 | 47.54 | 100.6 ± 17.6 |
| A2 | 106.2 ± 14.9 | 1.3 ± 0.1 | 1.7 ± 0.4 | — | 93.4 ± 16.5 |
| A3 | 92.7 ± 8.5 | 0.9 ± 0.2 | 1.5 ± 0.5 | — | 106.8 ± 20.8 |
| A4 | 82.8 ± 4.2 | 1.8 ± 0.7 | 1.5 ± 0.3 | — | 97.7 ± 20.5 |
| A5 | 55.2 ± 5.7 | 2.7 ± 0.9 | 0.9 ± 0.2 | 28.7 | 87.2 ± 19.3 |
| B1 | 105.2 ± 1.6 | 2.9 ± 1.7 | 2.1 ± 0.4 | 55.8 | 94.8 ± 17.6 |
| B2 | 94.4 ± 3.6 | 2.5 ± 0.7 | 1.3 ± 0.2 | — | 93.8 ± 18.6 |
| B3 | 87.7 ± 3.4 | 2.0 ± 0.8 | 0.8 ± 0.0 | — | 88.5 ± 19.4 |
| B4 | 86.7 ± 6.1 | 2.0 ± 0.4 | 1.3 ± 0.4 | — | 92.7 ± 20.0 |
| B5 | 75.6 ± 2.3 | 1.7 ± 0.3 | 1.0 ± 0.6 | 43.49 | 98.1 ± 20.7 |
| C1 | 107.5 ± 4.2 | 1.6 ± 0.3 | 1.6 ± 0.2 | 54.93 | 105.4 ± 17.5 |
| C2 | 94.1 ± 2.2 | 2.1 ± 0.2 | 1.6 ± 0.1 | — | 126.1 ± 19.2 |
| C3 | 88.3 ± 1.4 | 1.8 ± 0.5 | 1.4 ± 0.3 | — | 120.2 ± 27.9 |
| C4 | 86.4 ± 4.2 | 1.4 ± 0.3 | 1.1 ± 0.3 | — | 101.6 ± 22.0 |
| C5 | 74.4 ± 1.8 | 1.2 ± 0.1 | 0.8 ± 0.1 | 34 | 130.7 ± 33.9 |
| C6 | 109.2 ± 2.8 | 2.2 ± 0.8 | 1.9 ± 0.5 | 52.26 | 106.3 ± 18.1 |
| C7 | 102.1 ± 10.5 | 2.0 ± 0.6 | 1.6 ± 0.4 | — | 165.9 ± 46.2 |
| C8 | 92.2 ± 9.1 | 1.5 ± 0.2 | 1.1 ± 0.2 | — | 126.1 ± 19.2 |
| C9 | 80.1 ± 5.2 | 1.7 ± 0.3 | 1.1 ± 0.2 | — | 137.7 ± 26.3 |
| C10 | 80.4 ± 15.3 | 1.5 ± 0.2 | 0.8 ± 0.1 | 45.27 | 128.5 ± 23.3 |
| D1 | 105.7 ± 2.3 | 1.4 ± 0.2 | 2.0 ± 0.1 | 54.66 | 115.9 ± 24.3 |
| D2 | 103.2 ± 8.4 | 1.9 ± 0.8 | 1.4 ± 0.4 | — | 137.0 ± 29.4 |
| D3 | 91.5 ± 3.1 | 1.8 ± 0.3 | 1.4 ± 0.2 | — | 121.2 ± 22.8 |
| D4 | 79.4 ± 4.8 | 2.3 ± 0.6 | 1.5 ± 0.4 | — | 140.6 ± 36.1 |
| D5 | 72.4 ± 6.6 | 5.1 ± 0.7 | 1.2 ± 0.3 | 46.47 | 219.2 ± 54.4 |
| D6 | 122.1 ± 13.4 | 2.4 ± 0.3 | 2.3 ± 0.1 | 56.54 | 116.8 ± 17.7 |
| D7 | 120.5 ± 4.1 | 1.7 ± 0.1 | 1.8 ± 0.2 | — | 127.1 ± 20.1 |
| D8 | 100.3 ± 9.7 | 3.2 ± 1.4 | 2.9 ± 0.2 | — | 120.9 ± 18.7 |
| D9 | 77.5 ± 5.1 | 1.7 ± 0.4 | 1.0 ± 0.2 | — | 198.7 ± 31.9 |
| D10 | 73.9 ± 3.7 | 4.4 ± 0.9 | 2.3 ± 0.4 | 46.97 | 202.5 ± 37.8 |
| E1 | 108.0 ± 9.7 | 5.1 ± 2.1 | 4.5 ± 1.7 | 58.7 | 100.8 ± 22.9 |
| E2 | 103.0 ± 6.9 | 2.6 ± 0.4 | 2.2 ± 0.4 | — | 97.4 ± 21.7 |
| E3 | 96.4 ± 5.4 | 3.6 ± 1.5 | 2.4 ± 1.0 | — | 106.5 ± 25.9 |
| E4 | 94.7 ± 2.4 | 4.5 ± 1.6 | 3.1 ± 1.0 | — | 108.2 ± 29.2 |
| E5 | 95.0 ± 2.7 | 1.8 ± 0.0 | 1.4 ± 0.5 | 45.48 | 113.7 ± 29.0 |
| E6 | 107.0 ± 2.4 | 2.9 ± 0.7 | 2.3 ± 0.6 | 57.18 | 105.9 ± 26.4 |
| E7 | 100.5 ± 3.1 | 4.2 ± 0.8 | 3.0 ± 0.5 | — | 100.4 ± 30.3 |
| E8 | 102.8 ± 2.5 | 6.4 ± 4.3 | 4.3 ± 2.8 | — | 96.6 ± 21.6 |
| E9 | 116.9 ± 11.9 | 3.1 ± 0.7 | 1.6 ± 0.5 | — | 94.4 ± 24.3 |
| E10 | 121.6 ± 14.2 | 3.6 ± 0.1 | 2.8 ± 0.2 | 56.13 | 84.4 ± 20.0 |
| F1 | 106.5 ± 4.4 | 4.1 ± 1.0 | 2.1 ± 0.0 | 53.5 | 81.4 ± 17.7 |
| F2 | 105.8 ± 0.7 | 3.1 ± 0.2 | 2.4 ± 0.2 | — | 184.9 ± 20.4 |
| F3 | 115.6 ± 9.9 | 3.0 ± 0.9 | 2.5 ± 0.3 | — | 105.9 ± 26.4 |
| F4 | 116.2 ± 3.5 | 5.8 ± 0.8 | 3.0 ± 0.3 | — | 78.7 ± 17.3 |
| F5 | 110.1 ± 16.9 | 4.0 ± 0.3 | 3.1 ± 0.2 | 55.91 | 86.2 ± 17.3 |
| F6 | 110.1 ± 7.5 | 2.2 ± 0.3 | 1.9 ± 0.4 | 53.09 | 85.6 ± 16.1 |
| F7 | 95.0 ± 4.4 | 2.5 ± 0.6 | 1.9 ± 0.2 | — | 82.5 ± 15.3 |
| F8 | 81.8 ± 2.7 | 2.3 ± 0.2 | 1.7 ± 0.1 | — | 82.2 ± 14.5 |
| F9 | 71.4 ± 3.5 | 2.5 ± 0.0 | 1.4 ± 0.2 | — | 85.9 ± 19.3 |
| F10 | 71.9 ± 1.6 | 4.0 ± 0.2 | 2.1 ± 0.1 | 47.66 | 102.1 ± 23.6 |

A general trend among all the samples was that increasing the blend ratio from 10/90 to 50/50 decreased melting enthalpy and tensile modulus, indicating lower crystallinity of the materials, likely due to the increasing amorphous HEMA content. However, when the grafted PLLA chain length in PHEMA-g-PLLA-ma was long enough (e.g., 132,000 g/mol), the tensile modulus no longer significantly decreased with increasing blend ratio, indicating the effect of the graft PLLA chain length on the mechanical properties. It was likely that the long PLLA graft chain contributed to crystallinity and overall mechanical properties, where the polymer blend maintains similar physical properties as pure PLLA even at high PHEMA-g-PLLA-ma/PLLA blend ratio. This was corroborated by the melt enthalpy data.

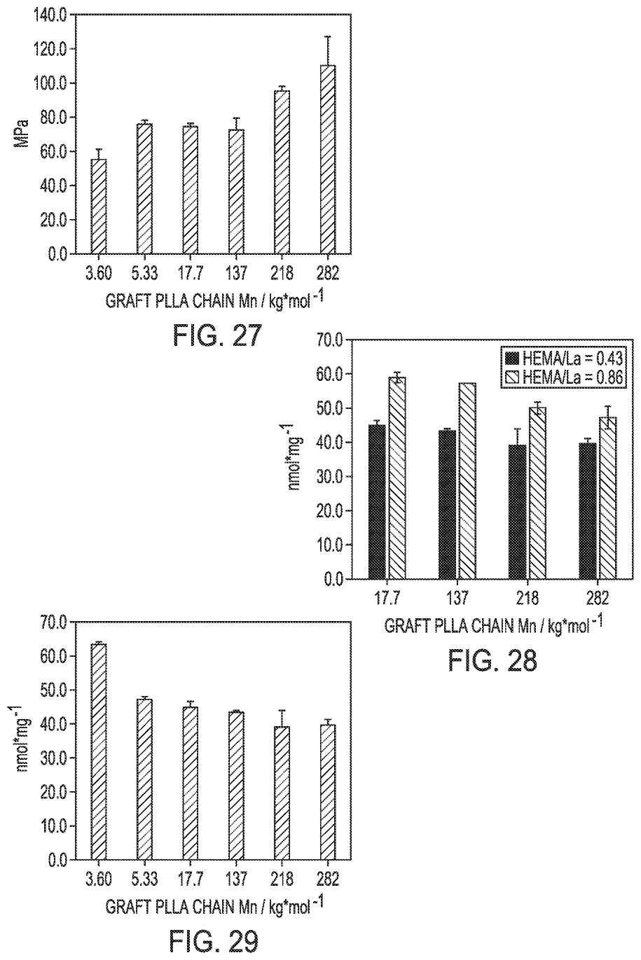

As shown in Table 2, the trend of modulus was similar against the HEMA/L-lactide feed ratio, i.e., the modulus decreased with increasing HEMA/L-lactide ratios. The modulus results for the 50/50 blend versus the graft PLLA chain length are shown in FIG. 27. As illustrated, increasing graft PLLA chain length resulted in higher modulus, possibly due to improved crystallization of PLLA (higher crystallinity and larger crystal size). These results were corroborated by the trend of increasing melting enthalpy when the graft PLLA chain was longer.

Overall, the example films exhibited desirable mechanical properties and nanofiber properties for the polymeric structures disclosed herein.

Nanofibrous 3D Porous Scaffold Fabrication with PHEMA-g-PLLA-ma/PLLA Blends

3D scaffolds were fabricated using the various weight ratios of PHEMA-g-PLLA-ma and PLLA (10/90, 20/80, 30/70, 40/60, and 50/50) following the procedure described in Example 2. These blend ratios were selected to achieve nanofibrous scaffolds. The porous 3D nanofibrous scaffolds were collected, cut into desired shapes and freeze dried until further experiments.

For polymeric structures that are to be used in bone regeneration, modulus and peptide conjugation density are important.

Higher modulus can better maintain pore shape and size, which are known to affect vascularization and osteogenic differentiation. Because the same blends were used for both film and scaffold formation, the mechanical properties and fiber properties shown in Table 2 are expected to be similar for the scaffolds.

The conjugation density determines how much peptide can interact with surface receptors and regulate cell behavior. The scaffolds were used to test peptide conjugation.

### Peptide Conjugation on Nanofibrous Films and 3D Porous Scaffolds

A piece of 5-mm-diameter 3D porous scaffold was weighted to calculate peptide conjugation amount per mass. The piece was wet with ethanol in a cell culture plate for 30 minutes before reaction. 1:1 volume of two solutions, i) 5 wt % IRGACURE® 2959/methanol and ii) from 0.1 to 1 mg/ml peptide (GVM2, GER2, or GER3)/PBS, were added to the 3D scaffolds, for a volume of 0.2 mL per piece. The varied peptide concentration was used to evaluate the effects of varying the peptide feed. The plate was moved under a UV lamp and exposed to UV light for 15 minutes. Supernatant was collected before and after reaction for peptide consumption measurements, and the 3D scaffold was washed with ethanol 3 times and PBS 3 times for 30 minutes each, and stored at −80 °C until further experiments.

The amount of conjugated peptide (μg peptide per mg scaffold) was determined by subtracting remaining peptide and wash-away peptide from total feeding peptide. The conjugation percentage was given by the ratio of conjugated peptides to total feeding peptides. Concentrations were determined following the protocol provided by Pierce™ Quantitative Peptide Assays (Thermo Fisher Scientific) and were converted into mole amount of peptide per mass scaffold. The results are shown in Table 3. The various scaffolds are identified by the blend used to form the scaffold.

TABLE 3

| | Peptide |
|---|---|
| Blend | Conjugation |
| ID | (nmol/mg) |
| A1 | 41.6 ± 0.1 |
| A2 | 49.8 ± 0.1 |
| A3 | 52.3 ± 0.2 |
| A4 | 58.3 ± 3.6 |
| A5 | 63.5 ± 0.6 |
| B1 | 27.2 ± 0.5 |
| B2 | 30.9 ± 0.1 |
| B3 | 28.7 ± 0.5 |
| B4 | 39.2 ± 3.0 |
| B5 | 47.1 ± 0.8 |
| C1 | 27.5 ± 0.9 |
| C2 | 28.1 ± 0.9 |
| C3 | 36.8 ± 1.3 |
| C4 | 37.6 ± 1.5 |
| C5 | 45.0 ± 1.6 |
| C6 | 33.5 ± 1.5 |
| C7 | 35.3 ± 0.8 |
| C8 | 38.5 ± 1.9 |
| C9 | 39.3 ± 0.9 |
| C10 | 54.0 ± 1.2 |
| D1 | 27.7 ± 0.3 |
| D2 | 27.2 ± 0.1 |
| D3 | 39.2 ± 0.3 |
| D4 | 35.4 ± 0.4 |
| D5 | 43.4 ± 0.4 |
| D6 | 43.6 ± 9.2 |
| D7 | 29.6 ± 2.3 |
| D8 | 36.9 ± 0.2 |
| D9 | 38.5 ± 0.5 |
| D10 | 52.2 ± 0.1 |
| E1 | 27.7 ± 0.6 |
| E2 | 29.1 ± 0.3 |
| E3 | 32.8 ± 0.3 |
| E4 | 39.1 ± 0.2 |
| E5 | 39.1 ± 4.8 |
| E6 | 33.4 ± 0.4 |
| E7 | 33.0 ± 1.3 |
| E8 | 37.6 ± 0.4 |
| E9 | 36.0 ± 0.0 |
| E10 | 45.8 ± 1.6 |
| F1 | 26.1 ± 0.9 |
| F2 | 28.8 ± 1.3 |
| F3 | 32.7 ± 1.9 |
| F4 | 35.8 ± 1.2 |
| F5 | 39.6 ± 1.5 |
| F6 | 33.4 ± 0.9 |
| F7 | 34.0 ± 1.1 |
| F8 | 36.2 ± 2.1 |
| F9 | 40.5 ± 2.1 |
| F10 | 43.3 ± 3.0 |

In contrast to the melt enthalpy and tensile modulus results for the nanofibrous films, peptide conjugation density on the nanofibrous scaffolds increased with increasing HEMA/L-lactide ratio since each HEMA segment brings one binding site (i.e., the methacrylate end group). The increase of peptide conjugation density was not proportional to the blend ratio, likely due to the high crowdedness of binding sites (methacrylate groups) on the surface of the nanofibers. The crowdedness may have impeded full conversion of methacrylate groups to peptides, even though the actual number of binding sites was proportional to the blend ratio.

The trends of modulus (form films) and peptide conjugation density (for scaffolds) were similar against the HEMA/L-lactide feed ratio, i.e., the modulus decreased with increasing HEMA/L-lactide ratios, and the conjugation density increased with increasing HEMA/L-lactide ratios (comparing relevant data in Tables 2 and 3). At the 50/50 PHEMA-g-PLLA-ma/PLLA blend ratio and for the graft copolymer having different PLLA chain lengths, differences in conjugation density were observed between the two HEMA/L-lactide feed ratios (0.43 vs 0.86). These results are shown in FIG. 28. Increasing HEMA/PLLA feed ratio is beneficial to conjugation density because more HEMA units contribute to more conjugation sites (methacrylate groups). However, increasing graft PLLA chain length reduced peptide conjugation density (see FIG. 29). This trend could be due to the difference in the methacrylate groups available for the conjugation reaction. The methacrylate groups were present at each HEMA unit and at the end of graft PLLA chain. The shorter the graft PLLA chain, the more methacrylate groups per mass of graft copolymer, resulting in a higher conjugation density. In addition, the comparison of molecular weight between PHEMA-g-PLLA-ma and graft PLLA chain (tested for the films, shown in Table 2) confirmed that there were potentially more graft PLLA chains per graft copolymer when a shorter graft PLLA chain was used. For a longer graft PLLA chain, the total number of methacrylate groups was smaller, therefore the conjugation density decreased with the increasing chain length (FIG. 29).

The peptide conjugation was also confirmed by FTIR (results similar to FIG. 20).

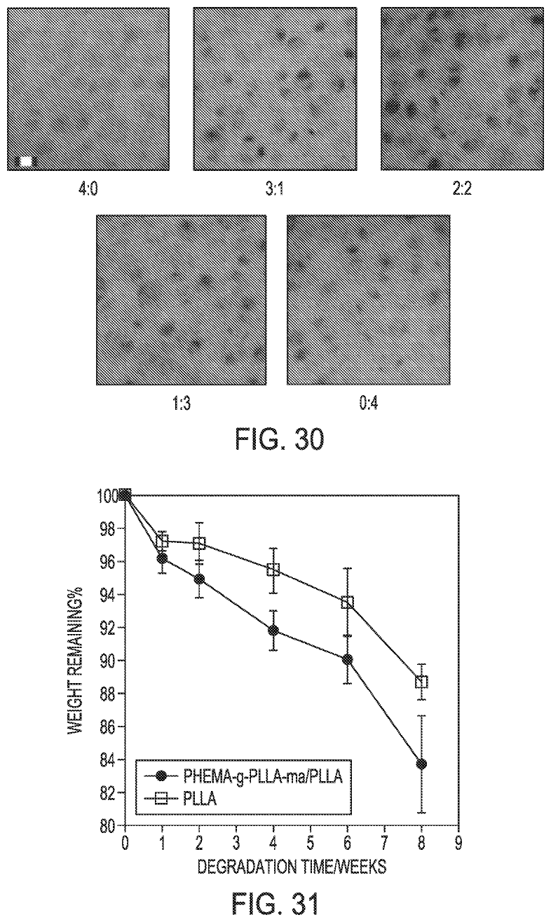

The GVM2 and GER3 peptides were labeled as described in Example 2. These peptides (FITC dye labeled GVM2) and (Avidin-Alex dye labeled GER3) were conjugated to the 3D scaffolds formed with Blend A2. For different scaffolds, the peptide ratio was varied during conjugation. The following ratios of GVM2 to GER3 were tested: 4:0, 3:1, 2:2, 1:3, and 0:4. To visualize the conjugation of the different peptides, a Leica Thunder microscope was used. The fluorescence images, which are reproduced herein in black and white, are shown in FIG. 30. The varying peptide content gave varying signal strength, which was dependent on the varying peptide feed ratio. These results suggest that the ratio of peptides on the base material (e.g., 3D scaffold) can be controlled by peptide feed ratio. The quantified results were similar to those shown in FIG. 24A and FIG. 24B.

Peptide conjugation quantification using the GER peptide alone on the A2 scaffold showed a conjugation amount of 1.6 nmol per mg scaffold, or 6.7 μg per mg of scaffold (0.67 wt %), when using a feeding of 4.0 nmol or 16.8 μg of peptide per mg of scaffold.

Conjugation (i.e., covalent attachment) was compared with physical absorption in terms of peptide retention in an aqueous solution over time. To prepare a physically absorbed example, GER3 was absorbed on a nanofibrous film (formed form Blend A2) at 4 °C overnight. This polymeric structure was compared with the Blend 2A scaffold covalently conjugated with GER3. The film and scaffold were placed in a PBS solution at 37 °C for up to 3 weeks with PBS changed every 2 days. The amount of remaining peptide was visualized by the intensity of fluorescence after staining with Streptavidin-Alexa Fluor 555 conjugate. While these results are not reproduced herein, after 3 weeks and 1 day, the remaining peptide on the physical adsorption group decreased substantially, while the fluorescence intensity from the covalently conjugated group did not change significantly. The results confirmed that covalent conjugation of peptide and its robustness over time.

Overall, the results for the films and scaffolds illustrate that when the blend ratio is fixed (e.g., at 50/50), higher HEMA content improves peptide conjugation capacity, higher PLLA content in the graft copolymer improves mechanical properties, longer graft PLLA chain length contributes to better mechanical properties, and shorter graft PLLA chains contribute to higher peptide conjugation properties. Balancing these factors can generate films and scaffolds with desirable properties for the biological compositions.

### Scaffold Degradation

Blend A2 3D porous scaffolds and pure PLLA scaffolds were prepared for this comparison.

The Blend A2 3D porous scaffolds and the pure PLLA scaffolds were cut into disks with a diameter of 18 mm and a thickness of 2 mm and were soaked in PBS at 37 °C for up to 8 weeks. 2 to 3 disks were used for each replicate and 3 replicates per timepoint were used for weight loss calculation. SEM images (not reproduced herein) were taken at 3 timepoints: 0 week (before incubation), 2 weeks and 8 weeks of incubation, to examine surface morphology changes. The SEM images indicated that pore surfaces of both types of scaffolds became more porous at 2 weeks and lost mechanical integrity at 8 weeks. However, the PHEMA-g-PLLA-ma/PLLA blend scaffolds degraded faster than those of pure PLLA scaffolds (see FIG. 31). Faster degradation is desirable for in vivo applications. The mass loss of the PHEMA-g-PLLA-ma/PLLA blend scaffolds was substantially faster than those of pure PLLA scaffolds. The mass loss data of the blend scaffolds at every time point (from 1 to 8 weeks) were statistically significantly lower than those of the control pure PLLA scaffolds (p<0.001). The HEMA portion of the polymer was more hydrophilic and amorphous, contributing to a faster degradation rate compared to pure PLLA even when 80% of the polymer blend was pure PLLA.

### Biocompatibility

For further examination in vitro and in vivo, scaffolds formed with Blend A2 were used.

To examine biocompatibility, 10k mouse bone marrow-derived mesenchymal stem cells (BMSCs) were seeded into each Blend A2 scaffold. More specifically, the mouse BMSC cells were harvested from legs of 6-to-8-week-old C57BL/6J mice and seeded on Blend A2 3D scaffolds (5 mm diameter, 2 mm thickness). The scaffolds were wetted with ethanol 3 times, PBS 3 times, and α-MEM one time at a density of 10k cells per scaffold.

The cell-seeded scaffolds were incubated with cell culture medium (α-MEM with 10% FBS) at 37 °C for up to 3 weeks. Samples were collected at 4 hours, 1 week, 2 weeks and 3 weeks after seeding. 150 uL Tris-NaCl-EDTA (TNE) buffer and 10 uL proteinase K (20 mg/mi) were used to remove protein at 55 °C for 24 hours and was later precipitated by adding 6M NaCl solution. Supernatants were measured following the protocol of Quant-iT™ dsDNA Assay Kits. The cell number of each sample was determined by comparing the DNA concentration to cell standards with the number of cells acquired by hemocytometer. The cell number increased with culture time, reaching about 4.6 times of the seeded cell number at 3 weeks. This indicated that the Blend A2 scaffold had good cell compatibility and supported cell proliferation.

### In Vivo Bone Regeneration

C57BL/6J mice of 6-to-8-week-old were pre-injected with 5 mg/kg carprofen and anesthetized with isoflurane. Each skull area was shaved and cleaned with iodine and saline solution. A 5-mm incision was made at the center line of each skull and a 5 mm-diameter trephine bur was used to carefully remove a round piece of bone. Cell-free Blend A2 3D scaffolds, with or without peptide conjugation and wetted with saline, were placed into respective defects and silk suture was used to close the incisions. The cell-free Blend A2 3D scaffold without peptides was the control sample. For the respective peptide conjugated samples, the following peptide concentrations were used: 100 μg GER2 per scaffold, 100 μg GVM2 per scaffold, and 100 μg GER2+100 μg GVM2 per scaffold. The proteins were conjugated to the scaffolds as described herein. The mice were sacrificed at 4-week and 8-week time points. Skulls were collected and underwent fixation in 4% paraformaldehyde at 4 °C for 24 hours. The samples were stored in 70% ethanol before characterization. Six animals were used for each group.

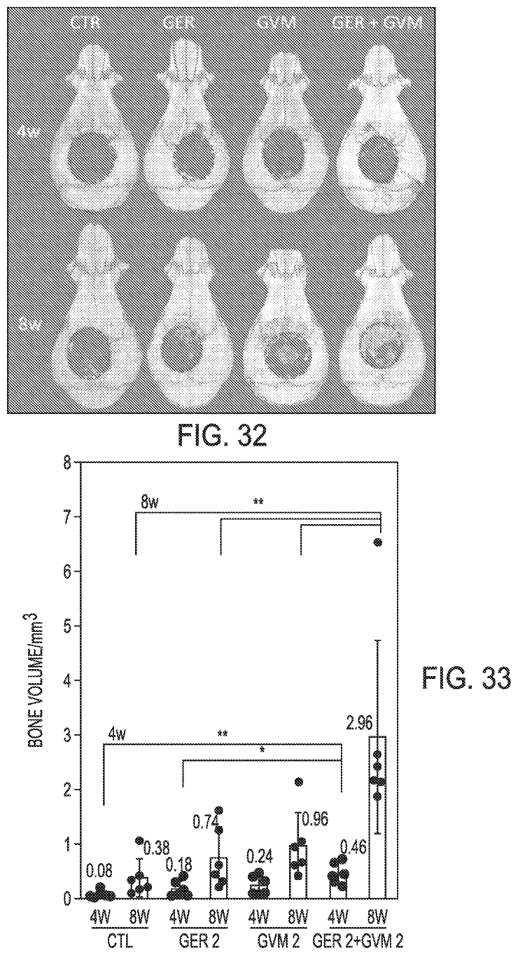

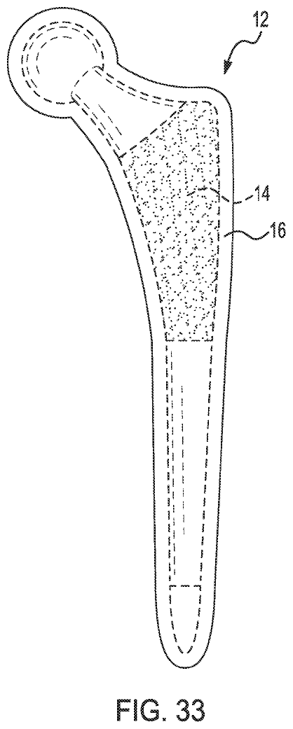

Regenerated bone volume was measured in the center of defects using micro-CT images. A micro-CT system (μCT100 Scanco Medical, Bassersdorf, Switzerland) was used to scan the samples under voxel size 18 μm, 70 kVp, 114 μA, 0.5 mm AL filter, and integration time of 500 ms. FIG. 32 illustrates black and white reproductions of the micro-CT images (where CTR=control; GER=GER2; GVM=GVM2; and GER+GVM=GER2+GVM2). The results were processed using dragonfly software to calculate the bone volume in the defects. FIG. 33 depicts these results.

Each peptide group showed significantly more bone volume between their own 4-week and 8-week time points, suggesting continuous bone regeneration over the time. At 8 weeks, there were significant differences (p<0.05) in bone volume between the GER2+GVM2 group and other groups, control, GER2, and GVM2. At 4 weeks, there were significant differences (p<0.05) in bone volume between the GER2+GVM2 group and two other groups, GER2 and control. The bone volumes of GER2+GVM2 groups were not only the highest among all groups, but also higher than the additive effect of two single peptides (GER2 or GVM2). At 4 weeks, the bone volume of the GER2+GVM2 group was 6.0 times the control group, which was equal to 2.6 times of GER2 alone, 1.9 times of GVM2 alone, and 1.1 times of the sum of GER2 and GVM2. At 8 weeks, the bone volume of the GER2+GVM2 group was 7.8 times the control group, which was equal to 4.0 times of GER2 alone group, 3.1 times of GVM2 alone, and 1.7 time of the sum of GER2 and GVM2 alone groups.

After the micro-CT scan, the samples were cleaned by removing soft tissues around the scaffold and demineralized in 14 wt % EDTA solution (pH=7.4) for 10 days. The samples were then embedded in paraffin, sliced and stained following the standard protocol of H&E staining and Masson's trichrome staining. While the results are not reproduced herein, the histology images showed similar trend as micro-CT images.

The biological compositions set forth herein include collagen-derived triple-helical peptides that selectively stimulate DDR signaling, or both DDR and β1 integrin signaling. As illustrated in the Examples, these peptides cooperatively interact to stimulate osteoblast differentiation of skeletal progenitor cells. The DDR-activating triple-helical peptide—mimicking the GVMGFO sequence present in fibrillar collagens—stimulated DDR2 phosphorylation and osteoblast differentiation of skeletal progenitor cells. The integrin-activating sequence—GFOGER—stimulated integrin signaling (FAK phosphorylation) as well as osteoblast differentiation. Significantly, when cells were exposed to both peptides, enhancements of DDR2 and β1 integrin signaling and synergistic stimulation of osteoblast differentiation were observed. As shown by gene deletion studies in primary cultures of calvarial osteoblasts, DDR2 was needed for the response to the DDR-activating GVMFGO peptide, and was also required for full stimulation of integrin signaling by the GFOGER peptide. As such, the biological compositions disclosed herein enable unique bone regenerative responses.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range. For example, a weight ratio ranging from 15:75 to 60:40 should be interpreted to include not only the explicitly recited limits of 15:75 to 60:40, but also to include individual weight ratios, and sub-ranges of weight ratios. Furthermore, when “about” is utilized to describe a value, this is meant to encompass minor variations (up to +/−10%) from the stated value.

In describing and claiming the examples disclosed herein, the singular forms “a”, “an”, and “the” include plural referents unless the context clearly dictates otherwise.

While several examples have been described in detail, it is to be understood that the disclosed examples may be modified. Therefore, the foregoing description is to be considered non-limiting.

1. A biological composition, comprising:
a base material; and
a peptide attached to a surface of the base material, wherein the peptide is to bind to a discoidin domain receptor of a cell.

2. The biological composition as defined in claim 1, further comprising a second peptide attached to the surface of the base material, wherein the second peptide is to bind to an integrin.

3. The biomaterial composition as defined in claim 2, wherein:
the peptide has SEQ. ID. NO. 2; and
the second peptide has SEQ. ID. NO. 3.

4. The biomaterial composition as defined in claim 2, wherein a total conjugation of the peptide and the second peptide is at least 4.4 nmol peptides/square meter base material surface area.

5. The biological composition as defined in claim 1, wherein the base material is selected from the group consisting of a polymer and a hydrogel.

6. The biological composition as defined in claim 5, wherein the polymer is one of:
a non-degradable polymer selected from the group consisting of polyethylene terephthalate, polystyrene, a silicone polymer, a polyurethane, polyetherether ketone, a polyamide, polycarbonate, (polytrimethylene carbonate), and mixtures thereof; or
a biodegradable polymer selected from the group consisting of poly(L-lactic acid) (PLLA), polyglycolic acid (PGA), poly(lactide-co-glycolide) (PLGA), poly(D,L-lactic acid) (PDLLA), polyanhydrides, poly(ortho ethers), poly(F-caprolactone) (PCL), poly(hydroxy butyrate) (PHB), poly(propylene fumarate) (PPF), polyphosphoesters (PPE), polyphosphazenes, polycarbonates, polyethylene glycol (PEG), polyvinyl alcohol (PVA), polyurethane, collagen, gelatin, elastin, alginate, chitin, chitosan, pectin, copolymers thereof, and mixtures thereof.

7. (canceled)

8. The biological composition as defined in claim 5, wherein the polymer is in a form of a structure selected from the group consisting of a scaffold, a film, and a microsphere.

9. The biomaterial composition as defined in claim 8, wherein the structure is nanofibrous.

10. The biomaterial composition as defined in claim 9, wherein the polymer includes a blend of poly(hydroxyethyl-methacrylate)-graft-poly(L-lactide)-methacrylate and poly(L-lactide) at a weight ratio ranging from 10:90 up to 60:40, wherein a tensile modulus of the structure is at least 10 kPa.

11. (canceled)

12. The biomaterial composition as defined in claim 5, wherein the polymer includes a blend of:
i) a nanofibrous polymer selected from the group consisting of poly(L-lactic acid), polycaprolactone, poly(lactide-co-glycolide) with either less than 15% lactide or less than 15% glycolide, poly(D,L-lactic acid), and combinations thereof, and
ii) a peptide conjugation polymer.

13. The biomaterial composition as defined in claim 5, wherein:
the polymer includes a blend of poly(hydroxyethyl-methacrylate)-graft-poly(L-lactide)-methacrylate and poly(L-lactide) at a weight ratio ranging from 10:90 to 60:40;
the poly(hydroxyethyl-methacrylate)-graft-poly(L-lactide)-methacrylate has a hydroxyethyl-methacrylate/L-lactide weight ratio ranging from 0.1 to 0.9; and
a number average molecular weight of the poly(L-lactide) is greater than or equal to 2,000 g/mol.

14. The biomaterial composition as defined in claim 5, wherein the polymer is poly(hydroxyethyl-methacrylate)-graft-poly(L-lactide)-methacrylate having a hydroxyethyl-methacrylate content ranging from 1 wt % to 99 wt %.

15. The biomaterial composition as defined in claim 1, wherein one of:
the peptide is attached to the surface by a covalent, ionic, or hydrogen bond; or
the peptide is attached to the surface via Van der Walls interactions.

16.-18. (canceled)

19. A method for forming a biomaterial composition, comprising attaching a peptide to a surface of a base material, wherein the peptide is to bind to a discoidin domain receptor of a cell.

20. The method as defined in claim 19, wherein:
the base material is a polymer;
the polymer is in a form of a scaffold; and
the method further comprises forming the scaffold by:
generating a negative replica of the scaffold by:
introducing sugar spheres and a non-solvent thereof into a mold;
annealing the sugar spheres, thereby causing the sugar spheres to interconnect; and
removing the non-solvent;
generating the scaffold by:
casting a polymer solution into the mold and onto the negative replica of the scaffold;
performing temperature-induced phase separation of the polymer solution; and
removing the negative replica of the scaffold.

21. The method as defined in claim 19, wherein attaching peptide to the surface of the base material involves:
wetting the base material;
exposing the wetted base material to the peptide, thereby forming a precursor structure; and
exposing the precursor structure to ultraviolet light.

22. The method as defined in claim 19, wherein attaching the peptide to the surface of the base material involves incubating, at a predetermined temperature and for a predetermined time, the base material in a solution containing the peptide.

23. The method as defined in claim 19, wherein:
the base material is a polymer blend of poly(hydroxyethyl-methacrylate)-graft-poly(L-lactide)-methacrylate and poly(L-lactide) at a weight ratio ranging from 10:90 up to 60:40; and
the method further comprises preparing the blend.

24. The method as defined in claim 19, further comprising attaching a second peptide to the surface of the base material, wherein:
the second peptide is to bind to an integrin;
the peptide has SEQ. ID. NO. 2; and
the second peptide has SEQ. ID. NO. 3.

25. (canceled)

26. The method as defined in claim 19, wherein:
the base material is a polymer;
the polymer is in a form of a film; and
forming the film involves thermally induced phase separation or electro-spinning.

27. The method as defined in claim 19, wherein:
the base material is a polymer;
the polymer is in a form of a microsphere; and
forming the microsphere involves an emulsion technique.


SEQUENCE LISTING (6 sequences)

SEQ ID NO: 1 - protein, 6 aa, Homo sapiens
GVMGFO

SEQ ID NO: 2 - protein, 12 aa, synthetic construct
  MOD_RES 12: note = 4Hyp
  MOD_RES 9: note = Nle
  MOD_RES 6: note = 4Hyp
GPRGQXGVXGFX

SEQ ID NO: 3 - protein, 6 aa, Homo sapiens
  MOD_RES 3: note = 4Hyp
GFXGER

SEQ ID NO: 4 - protein, 48 aa, synthetic construct
  MOD_RES 24: note = 4Hyp
  MOD_RES 27: note = Nle
  MOD_RES 30: note = 4Hyp
GPCGPPGPPGPPGPPGPPGPRGQXGVXGFXGPPGPPGPPGPPGPPGPC

SEQ ID NO: 5 - protein, 42 aa, synthetic construct
  MOD_RES 21: note = 4Hyp
GPCGPPGPPGPPGPPGPPGFXGERGPPGPPGPPGPPGPPGPC

SEQ ID NO: 6 - protein, 42 aa, synthetic construct
  MOD_RES 21: note = 4Hyp
GGPGPPGPPGPPGPPGPPGFXGERGPPGPPGPPGPPGPPGPC